(19) United States
(12) Patent Application Publication
(10) Pub. No.: US 2019/0142775 A1
(43) Pub. Date: May 16, 2019

(54) TREATING ACTIVATED DERMAL CONDITIONS WITH AGENTS THAT TARGET ENERGY METABOLISM

(71) Applicant: Sciaderm, Inc., Paoli, PA (US)

(72) Inventor: Klaus THEOBALD, Paoli, PA (US)

(21) Appl. No.: 16/098,157

(22) PCT Filed: May 2, 2017
(86) PCT No.: PCT/US2017/030528

§ 371 Date: Nov. 1, 2018

Related U.S. Application Data
(60) Provisional application No. 62/331,405, filed on May 3, 2016.

(51) Int. Cl. A61K 31/19; A61P 17/06; A61K 9/00; A61P 17/12

(52) U.S. Cl. CPC A61K 31/19; A61P 17/12; A61K 9/0014; A61P 17/06

(57) ABSTRACT

The present invention relates to methods of treating certain activated skin disorders in a subject comprising administering to the subject an effective amount of an agent that can reduce energy metabolism. An agent reduces energy metabolism may be administered topically as a dermatological product such as gels, creams, ointments, foams, patches or others.

### CROSS REFERENCE TO RELATED APPLICATIONS

This application claims the benefit of U.S. Provisional Application No. 62/331,405, filed May 3, 2016, the contents of which are herein incorporated by reference in its entirety.

### BACKGROUND

Many conditions affecting the outer or inner surfaces of living creatures such as skin or mucous membranes exist. Skin conditions can have diverse symptoms and in many cases the causes are not known, making it difficult to effectively treat the condition. However, many dermal conditions are associated with cell activation and/or proliferation.

### SUMMARY

The present disclosure provides the insight that certain skin disorders may be amenable to therapy that targets energy production, and particularly to therapy comprising dermal administration of therapy. The present disclosure encompasses a recognition that dermal administration of one or more inhibitors of energy production (e.g., inhibitors of glycolysis, inhibitors of mitochondrial function, etc.) can provide effective therapy for certain skin disorders, such as skin disorders associated with increased cell activation, including proliferation, herein referred to as activated skin disorders. Among other things, the present disclosure identifies the source of a problem with certain existing therapeutic regimens recommended for treatment of activated skin disorders (e.g., psoriasis, warts, eczema, etc.) in that they fail to target energy production, for example targeting inflammation and/or immune processes instead.

In some embodiments, a therapy for treatment of an activated skin disorder comprises administering one or more compounds that targets energy production. In some embodiments, a therapy that targets energy production may include administration of an agent that impairs or inhibits glycolysis (an “anti-glycolytic agent”), an agent that impairs or inhibits mitochondrial function, or an agent that otherwise interferes with the energy (ATP) production of a cell. In some embodiments, a therapy for treatment of an activated skin disorder comprises administering an effective amount of an anti-glycolytic agent. In some embodiments, an anti-glycolytic agent is a hexokinase inhibitor. In some embodiments, an anti-glycolytic agent is a glyceraldehyde-3-phosphate dehydrogenase inhibitor. In some embodiments, a therapy for treatment of an activated skin disorder comprises administering an effective amount of an agent that impairs mitochondrial function or a mitochondrial poison.

Agents that target energy production (e.g., anti-glycolytic agents) have been recognized as potentially toxic to cells that are highly reliant on energy metabolism, such as cancer cells and/or microbial cells, while having little or minimal toxic effects on normal tissues. Therefore, agents that target energy production (e.g., anti-glycolytic agents) have been suggested as anti-cancer agents and antimicrobial agents. The present disclosure provides, among other things, a surprising recognition that agents that target energy production can be effective for treating certain skin disorders, with potentially minimal induction or no induction of cell death of the target tissue.

In some embodiments, a non-toxic or minimally toxic level of one or more agents that target energy production is effective to treat an activated skin disorder. In some embodiments, an agent that targets energy production (e.g., an anti-glycolytic agent), is administered in an amount insufficient to kill cells of a target tissue (e.g. cells found in skin, which may include activated and/or non-activated cells). In some embodiments, a therapy for treatment of an activated skin disorder comprises administering an effective amount of an agent that targets energy production (e.g., an anti-glycolytic agent), wherein the agent induces little or no cell death of the non-activated cells in a target tissue (e.g. cells found in skin). In some embodiments, a therapy for treatment of an activated skin disorder comprises administering an effective amount of an agent that targets energy production (e.g., an anti-glycolytic agent), wherein the agent induces little or no cell death of either activated or non-activated cells in a target tissue (e.g. cells found in skin). In some embodiments, treatment of an activated skin disorder comprises administering an agent that targets energy production (e.g. an anti-glycolytic agent), wherein the agent is administered in an amount insufficient to eradicate the activated cells in the target tissue. In some embodiments, an agent that targets energy production (e.g., an anti-glycolytic agent), is administered in an amount effective to inhibit cell proliferation. In some embodiments, an agent that targets energy production (e.g., an anti-glycolytic agent), is administered in an amount that is insufficient to inhibit cell proliferation (e.g., partially inhibits, minimally inhibits or does not inhibit cell proliferation).

In some embodiments, a skin disorder associated with activation of cells that may be amenable to therapy that targets energy production includes psoriasis, warts, including genital warts, hyperkeratosis, ichthyosis, keratoderma, lichen planus, eczema or atopic dermatitis, and cervical dysplasia. In some embodiments, a skin disorder that is amenable to treatment with an agent that targets cellular energy production is psoriasis.

In some embodiments, an agent that targets energy production is represented by the general formula:

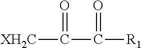

wherein X represents a halide, a sulfonate, a carboxylate, an alkoxide, or an amine oxide. In certain embodiments, X is a halide selected from the group consisting of: fluoride, bromide, chloride, and iodide. In some embodiments, X is a sulfonate. In certain embodiments, a sulfonate is selected from the group consisting of: triflate, mesylate and tosylate. In some embodiments, X is an amine oxide such as dimethylamine oxide. In certain embodiments, R1 represents OR, H, N(R″)2, C1-C6 alkyl, C6-C12 aryl, C1-C6 heteroalkyl, or a C6-C12 heteroaryl. Independently, in other embodiments, R″ represents H, C1-C6 alkyl, or C6-C12 aryl. Independently, in still other embodiments, R represents H, alkali metal, C1-C6 alkyl, C6-C12 aryl or C(O)R; and R′ represents H, C1-C20 alkyl or C6-C12 aryl. In some embodiments, an agent that targets energy production is a 3-halopyruvate. In certain embodiments, a 3-halopyruvate is selected from the group consisting of: 3-fluoropyruvate, 3-chloropyruvate, 3-bromopyruvate and 3-iodopyruvate. In one embodiment, a 3-halopyruvate is 3-bromopyruvate.

In certain embodiments, an agent that targets energy production is represented by the general formula:

X—CH₂—CO—COOH

wherein X represents a halide, a sulfonate, a carboxylate, an alkoxide, or an amine oxide. In some embodiments, X is a halide selected from the group consisting of: fluoride, bromide, chloride, and iodide. In some embodiments, X is a sulfonate is selected from the group consisting of: triflate, mesylate and tosylate. In some certain embodiments, X is an amine oxide such as dimethylamine oxide. In some embodiments, an agent that targets energy production is a 3-halopyruvate. In certain embodiments, a 3-halopyruvate is selected from the group consisting of: 3-fluoropyruvate, 3-chloropyruvate, 3-bromopyruvate and 3-iodopyruvate. In one embodiment, a 3-halopyruvate is 3-bromopyruvate. In some embodiments, an anti-glycolytic agent is an analog, derivative, prodrug, or metabolite of 3-bromopyruvate and salts thereof.

In some embodiments, an anti-glycolytic agent is or comprises a 3-halopyruvate. In certain embodiments, an anti-glycolytic agent is selected from the group consisting of: 3-fluoropyruvate, 3-chloropyruvate, 3-bromopyruvate and 3-iodopyruvate. In some embodiments, an anti-glycolytic agent is 3-bromopyruvate. In some embodiments, an anti-glycolytic agent is an analog, derivative, prodrug, or metabolite of 3-bromopyruvate or a salt thereof.

In some embodiments, an agent that targets energy production is selected from the group consisting of 2-deoxyglucose, 3-bromopyruvate, oxamate, apoptolidin and analogs, derivatives, prodrugs, metabolites or salts thereof.

In some embodiments, the present disclosure provides topical therapies for application to one or more surfaces in or on a subject. In some embodiments, one or more provided therapies is or comprises application to skin. In some embodiments, one or more provided therapies is or comprises application to a mucosal surface. In some embodiments, an agent that targets energy production is delivered in a topical formulation. In some embodiments, an agent that targets energy production is delivered in a topical formulation. In some embodiments, a topical formulation is or comprises an aqueous solution, a non-aqueous solution, a suspension, a cream, a lotion, a gel, or an ointment. In some embodiments, an agent that inhibits cellular energy production is formulated as a spray, mist, aerosol, solution, lotion, gel, cream, ointment, paste, unguent, emulsion or suspension.

In some embodiments, a topical formulation is or comprises an aqueous gel. In some embodiments, a topical formulation comprises an agent that targets energy production, a pharmaceutically acceptable carrier and a gelling agent. Gelling agents include carbomers, glycerine polyacrylate and mixtures thereof.

In some embodiments, a topical formulation is or comprises a cream or an ointment. In some embodiments, a topical formulation comprises an agent that targets energy production, a pharmaceutically acceptable carrier and further comprises: stearic acid, stearyl alcohol, cetyl alcohol, glycerin, and/or water.

In some embodiments, one or more provided topical therapies is or comprises application to skin. In some embodiments, one or more provided topical therapies is or comprises application to a mucosal surface. In some embodiments, a topical formulation comprises an agent that targets energy production in a concentration sufficient to decrease ATP production in a target tissue. In some embodiments, a topical formulation comprises an agent that targets energy production in a concentration insufficient to eradicate activated cells in a target tissue (e.g. cells in skin treated with a topical formulation). In some embodiments, a topical formulation comprises an agent that targets energy production in a concentration sufficient to inhibit cell proliferation in a target tissue (e.g. skin treated with a topical formulation). In some embodiments, a topical formulation comprises an agent that targets energy production in a concentration insufficient to inhibit cell proliferation or that minimally inhibits cell proliferation in a target tissue (e.g. cells in skin treated with a topical formulation).

In some embodiments, an agent that targets energy production is present in a topical formulation an amount in the range of about 0.0001 percent to about 50 percent of the total weight of the formulation. In some embodiments, an agent that targets energy production is present in a topical formulation in an amount within a range bounded by a lower limit and an upper limit, the upper limit being larger than the lower limit. In some embodiments, the lower limit may be about 0.001%, 0.002%, 0.005%, 0.01%, 0.02%, 0.05%, 0.1%, 0.2%, 0.5%, 1%, 2%, 5%, 10%, or 20%. In some embodiments, the upper limit may be about 0.005%, 0.01%, 0.02%, 0.05%, 0.1%, 0.2%, 0.5%, 1%, 2%, 5%, 10%, 20%, 30%, 40% or 50%.

In some embodiments, an agent that targets energy production is present in a topical formation as, for example, in an amount equal to or greater than about 20%, about 2%, about 0.2%, about 0.02%, or about 0.002% of the total weight of the formulation. In some embodiments, an agent that targets energy production may be present in a topical formulation in an amount below about 25%; in some embodiments, an agent that targets energy production may be present in a topical formulation in an amount above about 0.001%, 0.0025%, 0.0083%, 0.01%, 0.0167%, 0.02%, 0.03%, 0.04%, 0.05% or more; in some embodiments above about 0.1%, 0.2%, 0.3%, 0.4%, 0.5% or more; in some embodiments about 0.001%, 0.01%, 0.1%, 1%, 2%, 3%, 4%, 5%, or more. In some embodiments, an agent that targets energy production may be present in a topical formulation in an amount within a range of about 0.001% to about 50%, 0.01% to about 40%, 0.02% to about 30%, 0.03% to about 25%; in some embodiments within a range of about 0.0025% and about 5%; in some embodiments within a range of about 1% to about 20%, etc.

In some embodiments, a topical formulation of the present disclosure includes an agent that targets energy production at a concentration corresponding to about 0.001% to about 20% of 3-BP. In some embodiments, an agent that targets energy production is present in a topical formulation an amount within a range bounded by a lower limit and an upper limit, the upper limit being larger than the lower limit. In some embodiments, the lower limit corresponds to about 0.001%, 0.002%, 0.005%, 0.01%, 0.02%, 0.05%, 0.1%, 0.2%, 0.5%, 1%, 2%, 5%, 10%, or 20% of 3-BP. In some embodiments, the upper limit corresponds to about 0.005%, 0.01%, 0.02%, 0.05%, 0.1%, 0.2%, 0.5%, 1%, 2%, 5%, 10%, 20%, 30%, 40% or 50% of 3-BP. In some embodiments, a topical formulation of the present disclosure includes an agent that targets energy production at a concentration corresponding to about 0.0025% to about 5% of 3-BP. In some embodiments, a topical formulation of the present disclosure includes an agent that targets energy production at a concentration corresponding to about 0.004% to about 1.67% of 3-BP. In some embodiments, a topical formulation of the present disclosure includes an agent that targets energy production at a concentration corresponding to about 0.0083% of 3-BP. Those skilled in the art will appreciate that a concentration of a particular agent may be considered to be “corresponding to” a concentration of 3-BP if, at that concentration, the agent delivers or achieves a relevant result or activity that is comparable, in the system utilized, to that delivered or achieved by the stated concentration of 3-BP.

In some embodiments, a topical formulation of the present disclosure includes an agent that targets energy production at a concentration corresponding to about 0.15 mM to about 500 mM of 3-BP. In some embodiments, an agent that targets energy production is present in a topical formulation an amount within a range bounded by a lower limit and an upper limit, the upper limit being larger than the lower limit. In some embodiments, the lower limit corresponds to about 0.15 mM, 0.2 mM, 0.25 mM, 0.3 mM, 0.4 mM, 0.5 mM, 0.75 mM, 1 mM, 2, mM, 3 mM, 4 mM, 5 mM, 6 mM, 7 mM, 8 mM 9 mM, 10 mM, 20 mM, 30 mM, 40 mM 50 mM, 60 mM, 70 mM, 80 mM, 90 mM, 100 mM, or 200 mM of 3-BP. In some embodiments, the upper limit corresponds to about 0.25 mM, 0.3 mM, 0.4 mM, 0.5 mM, 0.75 mM, 1 mM, 2, mM, 3 mM, 4 mM, 5 mM, 6 mM, 7 mM, 8 mM 9 mM, 10 mM, 20 mM, 30 mM, 40 mM 50 mM, 60 mM, 70 mM, 80 mM, 90 mM, 100 mM, 200 mM, 300 mM, 400 mM, 500 mM of 3-BP. In some embodiments, a topical formulation of the present disclosure includes an agent that targets energy production at a concentration corresponding to about 0.25 mM to about 100 mM of 3-BP. In some embodiments, a topical formulation of the present disclosure includes an agent that targets energy production at a concentration corresponding to about 0.1 mM to about 10 mM of 3-BP.

In some embodiments, a preferred dose range of an agent that targets energy production may be within 0.0025% (0.15 mM) and 0.025% (1.5 mM). In some embodiments, preferred compositions comprising an agent that targets energy production are likely to be characterized by an absence or minimal amount of water present in the formulation.

In some embodiments, an agent that targets energy production is 3-bromopyruvate, 3-BP. In some embodiments, a preferred dose range of 3-BP may be within 0.0025% (0.15 mM) and 0.025% (1.5 mM). In some embodiments, preferred compositions comprising 3-BP are likely to be characterized by an absence or minimal amount of water present in the formulation.

In some embodiments, a topical formulation of an agent that targets energy production has a pH value in the range of about pH 2.5 to 8 or about pH 3 to 7, or about pH 3 to 6. In some embodiments, a topical formulation for an agent that targets energy production is has a pH value of approximately pH 3.

In some embodiments, a patient is administered 1 or more unit doses of a topical formulation of an agent that targets energy production. In some embodiments, a unit dose is about 54 to about 1 mL. In some embodiments, a unit dose is approximately, about 25 μL to about 50 μL.

In some embodiments, a topical formulation comprises an agent that targets energy production, a pharmaceutically acceptable carrier and further comprises a preservative. In some embodiments, a topical formulation comprises an agent that targets energy production, a pharmaceutically acceptable carrier and further comprises a local anesthetic. In some embodiments, a topical formulation comprises an agent that targets energy production, a pharmaceutically acceptable carrier and further comprises a skin humectant.

In some embodiments, an agent that inhibits cellular energy production is administered in combination with one or more additional agents to treat an activated skin disorder, such as an antibiotic, a retinoid, an anti-inflammatory agent, an immunosuppressive agent, a corticosteroid, vitamin A, a vitamin A derivative, vitamin D, or vitamin D analogs.

The present disclosure encompasses a recognition that topical administration of one or more anti-glycolytic agents can provide effective therapy for activated skin disorders, such as psoriasis. In some embodiments, a therapy treating or preventing reoccurrence of activated skin disorders, such as psoriasis, comprises administering an effective amount of an anti-glycolytic agent. In some embodiments, an anti-glycolytic agent is a 3-halopyruvate. 3-halopyruvate includes 3-fluoropyruvate, 3-chloropyruvate, 3-bromopyruvate and 3-iodopyruvate. In one embodiment, an anti-glycolytic agent is 3-bromopyruvate. In some embodiments, an anti-glycolytic agent is an analog, derivative, prodrug, or metabolite of 3-bromopyruvate or salts thereof. In some embodiments, an anti-glycolytic agent is a hexokinase inhibitor. Hexokinase inhibitors include 3-bromopyruvate and 2-deoxyglucose.

In some embodiments, the present disclosure provides topical therapies for application to one or more surfaces in or on a subject. In some embodiments, a topical composition comprises an anti-glycolytic agent and a pharmaceutically acceptable carrier. In some embodiments the anti-glycolytic agent is 3-bromopyruvate or a pharmaceutically acceptable salt thereof.

In some embodiments, the present disclosure provides topical therapies for application to one or more surfaces in or on a subject. In some embodiments, one or more provided therapies is or comprises application to skin. In some embodiments, administration of an anti-glycolytic agent reduces ATP production in the cells found in skin of a human subject. In some embodiments, cells found in the skin of a human subject may be, for example, skin cells such as keratinocytes, connective tissue cells such as fibrocytes, immune system cells such as lymphocytes or dendritic cells, and combinations thereof. Alternatively or additionally, in some embodiments, cells found in the skin of a human subject may be cells associated with other tissue types such as blood vessels, the nervous system, etc.

In some embodiments, a topical formulation comprises an anti-glycolytic agent in a concentration sufficient to decrease glucose metabolism in the skin of a subject treated with the topical composition. In some embodiments, a topical formulation comprises an anti-glycolytic agent in a concentration sufficient to decrease ATP production in a target tissue (e.g. cells in skin treated with a topical formulation, which may include activated and/or non-activated cells). In some embodiments, a topical formulation comprises an anti-glycolytic agent in a concentration insufficient to induce cell death in a target tissue (e.g. cells in skin treated with a topical formulation, which may include activated and/or non-activated cells). In some embodiments, a topical formulation comprises an anti-glycolytic agent in a concentration insufficient to induce cell death of activated and/or non-activated cells in a target tissue. In some embodiments, a topical formulation comprises an anti-glycolytic agent in a concentration insufficient to eradicate the activated cells in the target tissue. In some embodiments, a topical formulation comprises an anti-glycolytic agent in a concentration sufficient to inhibit cell proliferation in a target tissue (e.g. cells in skin treated with a topical formulation, which may include activated and/or non-activated cells). In some embodiments, a topical formulation comprises an anti-glycolytic agent in a concentration insufficient to inhibit or that minimally inhibits cell proliferation in a target tissue (e.g. cells in skin treated with a topical formulation).

In some embodiments, an anti-glycolytic agent is present in the topical formulation in an amount within the range of about 0.0001 percent to about 50 percent of the total weight of the formulation. In some embodiments, an anti-glycolytic agent is present in a topical formation within a range of, for example, 0.002% to 20%, or 0.002% to 2%, or 0.002% to 0.2%, or 0.002% to 0.02%, or 0.02% to 20%, or 0.02% to 2%, or 0.02% to 0.2%, or 0.2% to 20%, or 0.2% to 2%, or 2% to 20% of the total weight of the formulation. In some embodiments, an anti-glycolytic agent may be present in a topical formulation in an amount below about 25%; in some embodiments, an anti-glycolytic agent may be present in a topical formulation in an amount above about 0.01%, 0.02%, 0.03%, 0.04%, 0.05% or more; in some embodiments above about 0.1%, 0.2%, 0.3%, 0.4%, 0.5% or more; in some embodiments about 1%, 2%, 3%, 4%, 5%, or more. In some embodiments, an anti-glycolytic agent may be present in a topical formulation in an amount within a range of about 0.01% to about 50%, 0.1% to about 40%, 0.02% to about 30%, 0.03% to about 25%; in some embodiments within a range of about 0.05% and about 5%; in some embodiments within a range of about 1% to about 20%, etc.

In some embodiments, the anti-glycolytic agent is 3-bromopyruvate. In some embodiments, a topical formulation of the present disclosure includes 3-bromopyruvate at a concentration from about 0.0025% (0.15 mM) to about 5% (300 mM). In some embodiments, a topical formulation of the present disclosure includes 3-bromopyruvate at a concentration from about 0.005% (0.3 mM) to about 2.6% (156 mM). In some embodiments, a topical formulation of the present disclosure includes 3-bromopyruvate at a concentration of about 0.5 mM (0.0083%).

Those skilled in the art are aware that the term “pyruvate” is often used to refer to the relevant chemical compound in its solution form; solubilization of a “pyruvic acid” compound can be said to generate a “pyruvate” solution. Thus, a formulation that includes 3-bromopyruvate at a particular concentration (or molarity) may typically be prepared by combining 3-bromo-pyruvic acid with other components in the formulation to the stated concentration (or molarity). Those skilled in the art will therefore appreciate that “3-BP” as used herein may, as will be clear from context, refer to 3-bromopyruvate, which may have been utilized in the form of 3-bromo-pyruvic acid (“BPA”).

In some embodiments, a topical formulation has a pH value in the range of about pH 2.5 to 8 or about pH 3 to 7, or about pH 3 to 6.

In some embodiments, an anti-glycolytic agent is delivered in a topical formulation. In some embodiments, a topical formulation is an aqueous solution, a non-aqueous solution, a suspension, a cream, a lotion, a gel, or an ointment.

In some embodiments, an anti-glycolytic agent is formulated as a spray, mist, aerosol, solution, lotion, gel, cream, ointment, paste, unguent, emulsion or suspension.

In some embodiments, a topical formulation is or comprises an aqueous gel. In some embodiments, a topical formulation comprises an anti-glycolytic agent, a pharmaceutically acceptable carrier and a gelling agent. Gelling agents include carbomers, glycerine polyacrylate and mixtures thereof.

In some embodiments, a topical formulation is or comprises a cream or an ointment. In some embodiments, a topical formulation comprises an anti-glycolytic agent, a pharmaceutically acceptable carrier and further comprises: stearic acid, stearyl alcohol, cetyl alcohol, glycerin, and/or water.

In some embodiments, a topical formulation comprises an anti-glycolytic agent, a pharmaceutically acceptable carrier and further comprises a preservative. In some embodiments, a topical formulation comprises an anti-glycolytic agent, a pharmaceutically acceptable carrier and further comprises a local anesthetic. In some embodiments, a topical formulation comprises an anti-glycolytic agent, a pharmaceutically acceptable carrier and further comprises a skin humectant.

In some embodiments, the present disclosure provides technologies particularly suited for treatment or prevention of psoriasis, for example by administering an anti-glycolytic agent in combination with one or more additional agents such as an antibiotic, a retinoid, an anti-inflammatory agent, an immunosuppressive agent, a corticosteroid, vitamin A, a vitamin A derivative, vitamin D, a vitamin D analog, etc., or a combination thereof.

Analogously, in some embodiments, the present disclosure provides methods of manufacturing a medicament for topical delivery of an agent that targets energy production for the treatment or prevention of psoriasis, for example comprising combining an agent that inhibits ATP production and a pharmaceutically acceptable carrier. In some embodiments, the present disclosure provides methods of manufacturing a medicament for topical administration comprising an anti-glycolytic agent. In some embodiments, the present disclosure provides methods for manufacturing a medicament for topical administration comprising 3-bromopyruvate.

Also, in some embodiments, a medicament for topical delivery of an agent (e.g. an anti-glycolytic agent) for the treatment or prevention of psoriasis as described herein is an aqueous solution, a non-aqueous solution, a suspension, a cream, a lotion, a gel, or an ointment. In some embodiments, a medicament for topical delivery of an agent (e.g. an anti-glycolytic agent) for the treatment or prevention of psoriasis is formulated as a spray, mist, aerosol, solution, lotion, gel, cream, ointment, paste, unguent, emulsion or suspension.

In some embodiments, a topical medicament comprises an agent (e.g. an anti-glycolytic agent) at a concentration insufficient to eradicate activated cells in a target tissue (e.g. skin treated with a topical medicament). In some embodiments, a topical medicament comprises an agent (e.g. an anti-glycolytic agent) at a concentration sufficient to inhibit cell proliferation in a target tissue (e.g. skin treated with a topical formulation). In some embodiments, a topical medicament comprises an agent (e.g. an anti-glycolytic agent) at a concentration insufficient to inhibit or that minimally inhibits cell proliferation in a target tissue (e.g. skin treated with a topical formulation).

In some embodiments, a medicament for topical delivery of an agent that targets energy production for the treatment or prevention of psoriasis further comprises a preservative. In some embodiments, a medicament for topical delivery of an agent that targets energy production for the treatment or prevention of psoriasis further comprises a local anesthetic. In some embodiments, a medicament for topical delivery of an agent that targets energy production for the treatment or prevention of psoriasis further comprises a skin humectant.

In some embodiments, methods for treating an activated skin disorder are provided comprising administering a topical formulation of the present disclosure, wherein the treatment has been demonstrated to achieve a reduction in one or more symptoms of an activated skin disorder. In some embodiments, symptoms of an activated skin disorder include raised, red lesions covered by silvery white scales that show signs of hyperproliferation, inflammation. In some embodiments, a psoriatic plaque may itch and/or bleed easily. In some embodiments, one or more symptoms of an activated skin disorder are alleviated for a period of at least 2 weeks, 1 month, 3 months, at least 4 months, at least 5 months, at least 6 months, at least 7 months, at least 8 months, at least 9 months, at least 10 months, at least 11 months, at least one year, or more.

In some embodiments, treatment of an activated skin disorder with a topic formulation of the present disclosure may achieve a reduction in one or more symptoms of an activated skin disorder within 1 week of treatment, 2 weeks of treatment, 3 weeks of treatment, 4 weeks of treatment, 5 weeks of treatment, 6 weeks of treatment, 7 weeks of treatment, or 8 weeks of treatment. In some embodiments, treatment of an activated skin disorder with a topic formulation of the present disclosure has been demonstrated to achieve a reduction in one or more symptoms of an activated skin disorder within 2-6 weeks of treatment.

In some embodiments, treatment of an activated skin disorder with a topic formulation of the present disclosure may completely resolve one or more psoriatic lesions in a subject. In some embodiments, treatment of an activated skin disorder with a topic formulation of the present disclosure has been demonstrated to completely resolve 20%, 30%, 40%, 50%, 60%, 70%, 80%, 90% or more of the psoriatic lesions treated.

### BRIEF DESCRIPTION OF THE DRAWING

The Drawing included herein, which is composed of the following Figures, is for illustration purposes only and not for limitation.

FIG. 1 illustrates treatment effect on a first psoriatic plaque on the left forearm of a subject “before” and 20 weeks “after” last treatment with an exemplary 3-BP formulation. The left image was taken before the start of treatment and the right image was taken 26 weeks after baseline (i.e. 20 weeks after last treatment application.) FIG. 1 illustrates that plaques remain in complete remission even 20 weeks after last treatment application.

FIG. 2 illustrates treatment effect on a first psoriatic plaque on the right forearm of a subject at weekly intervals during treatment with an exemplary 3-BP formulation. Images show Day 1 (before treatment) and Days 7, 14 and 21 of treatment.

FIG. 3 illustrates a newly evolved plaque in a subject. The original plaque #2 is shown on the left panel and the newly evolved Plaque #3 is seen on the right, skin landmarks clearly shows the difference in location.

FIG. 4 illustrates treatment effect of a psoriatic plaque located on the elbow of a subject. The image, taken on Day 47 of treatment with an exemplary 3-BP formulation, shows the left (treated) elbow in comparison to the right (untreated) elbow.

### DEFINITIONS

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, nucleic acids (e.g., siRNAs, shRNAs, DNA/RNA hybrids, antisense oligonucleotides, ribozymes), peptides, peptide mimetics, etc.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Subject: As used herein, the term “subject” means any animal, including a mammal, to which will be or has been administered compounds or topical formulations of the present disclosure. The term “mammal” as used herein, encompasses any mammal. Examples of mammals include, but are not limited to, cows, horses, sheep, pigs, cats, dogs, mice, rats, rabbits, guinea pigs, monkeys, humans etc. Preferably the subject is a human.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or is susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes an activated skin disorder. In some embodiments, the patient is receiving or has received certain therapy to diagnose and/or to treat a disease, disorder, or condition.

Treatment: As used herein, the term “treatment” (also “treat” or “treating”) refers to any administration of a substance (e.g., an anti-glycolytic agent) that partially or completely alleviates, ameliorates, relives, inhibits, delays onset of, reduces severity of, and/or reduces incidence of one or more symptoms, features, and/or causes of a particular disease, disorder, and/or condition (e.g., psoriasis). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. In some embodiments, refers to an amelioration, prophylaxis, or reversal of a disease or disorder, or at least one discernible symptom thereof. For example, treating psoriasis or its symptoms by lessening the redness of the skin.

Prevention: As used herein, “prevention” (also “prevent” or “preventing”) when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time. In some embodiments, the compounds of the present disclosure are administered as a preventative measure to a subject having a predisposition to a skin disorder (e.g. psoriasis), even though symptoms of the disorder are absent or minimal.

Therapeutically effective amount: As used herein, a “therapeutically effective amount” of a compound of the present disclosure means the amount of the compound that is effective to treat or prevent recurrence of a condition or its signs or symptoms. The full therapeutic effect does not necessarily occur by administration of one dose, and may occur only after administration of a series of doses. Thus, a therapeutically effective amount may be administered in one or more administrations of a unit dosage form.

Prophylactically effective amount: As used herein, a “prophylactically effective amount” of a drug is an amount of a drug that, when administered to a subject, will have the intended prophylactic effect, e.g., preventing or delaying the onset or reoccurrence of disease or symptoms, or reducing the likelihood of the onset or reoccurrence of disease or symptoms. The full prophylactic effect does not necessarily occur by administration of one dose, and may occur only after administration of a series of doses. Thus, a prophylactically effective amount may be administered in one or more administrations.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent, involved in carrying or transporting an agent that targets energy production to an organ, or portion of the body, for example, a composition for topical administration may comprise a pharmaceutically acceptable carrier that is involved in (e.g., promotes and/or achieves) skin penetration. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Analog: The term “analog” refers to a chemical compound that is structurally similar to a parent compound and has chemical properties or pharmaceutical activity in common with the parent compound. Analogs include, but are not limited to, homologs, i.e., where the analog differs from the parent compound by one or more carbon atoms in series; positional isomers; compounds that differ by interchange of one or more atoms by a different atom, for example, replacement of a carbon atom with an oxygen, sulfur, or nitrogen atom; and compounds that differ in the identity of one or more functional groups, for example, the parent compound differs from its analog by the presence or absence of one or more suitable substituents. Suitable substituents include, but are not limited to, (C1-C8)alkyl; (C1-C8)alkenyl; (C1-C8)alkynyl: aryl; (C2-05)heteroaryl; (C1-C6)heterocycloalkyl; (C3-C7)cycloalkyl; O—(C1-C8)alkyl; O—(C1-C8)alkenyl; O—(C1-C8)alkynyl; O-aryl; CN; OH; oxo; halo, C(O)OH; COhalo; O(CO)halo; CF3, N3; NO2, NH2; NH((C1-C8)alkyl); N((C1-C8)alkyl)2; NH(aryl); N(aryl) N((C1-C8)alkyl)(aryl); (CO)NH2; (CO)NH((C1-C8)alkyl); (CO)N((C1-C8)alkyl)2; (CO)NH(aryl); (CO)N(aryl)2; O(CO)NH2; NHOH; NOH((C1-C8)alkyl); NOH(aryl); O(CO)NH((C1-C8)alkyl); O(CO)N((C1-C8)alkyl)2; O(CO)NH(aryl); O(CO)N(aryl)2; CHO; CO((C1-C8)alkyl); CO(aryl); C(O)O((C1-C8)alkyl); C(O)O(aryl); O(CO)((C1-C8)alkyl); O(CO)(aryl); O(CO)O((C1-C8)alkyl); O(CO)O(aryl); S—(C1-C8)alkyl; S—(C1-C8)alkenyl; S—(C1-C8)alkynyl; S-aryl; S(O)C1-C8)alkyl; S(O)—(C1-C8)alkenyl; S(O)—(C1-C8)alkynyl; and S(O)-aryl; S(O)2-(C1-C8)alkyl; S(O)2-(C1-C8)alkenyl; S(O)2-(C1-C8)alkynyl; and S(O)2-aryl. One of skill in the art can readily choose a suitable substituent based upon the stability and pharmacological activity of the compound of the present disclosure.

Alkyl: The term “alkyl” means a saturated, monovalent, unbranched or branched hydrocarbon chain. Examples of alkyl groups include, but are not limited to, (C1-C3)alkyl groups, such as methyl, ethyl, propyl, isopropyl and (C4-C8)alkyl groups, such as 2-methyl-1-propyl, 2-methyl-2-propyl, 2-methyl-1-butyl, 3-methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2pentyl, 4-methyl-2-pentyl, 2,2-dimethyl-1-butyl, 3,3 dimethyl-1-butyl, 2-ethyl-1-butyl, butyl, isobutyl, t-butyl, pentyl, isopentyl, neopentyl, and hexyl, heptyl, and octyl. An alkyl group can be unsubstituted or substituted with one or two suitable attachments.

Alkenyl: The term “alkenyl” means a monovalent, unbranched or branched hydrocarbon chain having one or more double bonds therein. The double bond of an alkenyl group can be unconjugated or conjugated to another unsaturated group. Suitable alkenyl groups include, but are not limited to (C2-C8)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)pentenyl. An alkenyl group can be unsubstituted or substituted with one or two suitable substituents.

Alkynyl: The term “alkynyl” means monovalent, unbranched or branched hydrocarbon chain having one or more triple bonds therein. The triple bond of an alkynyl group can be unconjugated or conjugated to another unsaturated group. Suitable alkynyl groups include, but are not limited to, (C2-C8)alkynyl groups, such as ethynyl, propynyl, butynyl, pentynyl, hexynyl, methylpropynyl, 4-methyl-butynyl, 4-propyl-2-pentynyl, and 4-butyl-2-hexynyl. An alkynyl group can be unsubstituted or substituted with one or two suitable substituents.

Aryl: The term “aryl” means a monocyclic or polycyclic aromatic group comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C6)aryl”.

Heteroaryl: The term “heteroaryl” means a monocyclic- or polycyclic aromatic ring comprising carbon atoms, hydrogen atoms, and one or more heteroatoms, preferably, 1 to 3 heteroatoms, independently selected from nitrogen, oxygen, and sulfur. As is well known to those skilled in the art, heteroaryl rings have less aromatic character than their all-carbon counter parts. Thus, for the purposes of the present disclosure, a heteroaryl group need only have some degree of aromatic character. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl, phenyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “(C2-05)heteroaryl”.

Cycloalkyl: The term “cycloalkyl” means a non-aromatic, monocyclic or polycyclic ring comprising carbon and hydrogen atoms. A cycloalkyl group can have one or more carbon-carbon double bonds in the ring so long as the ring is not rendered aromatic by their presence. Examples of cycloalkyl groups include, but are not limited to, (C3C7)cycloalkyl groups, such as cyclopropyl, cyclobutyl, cyclopentyl, cyclohexyl, and cycloheptyl, and saturated cyclic and bicyclic terpenes and (C3-C7)cycloalkenyl groups, such as cyclopropenyl, cyclobutenyl, cyclopentenyl, cyclohexenyl, and cycloheptenyl, and unsaturated cyclic and bicyclic terpenes. Acycloalkyl group can be unsubstituted or substituted by one or two suitable substituents. Preferably, the cycloalkyl group is a monocyclic ring or bicyclic ring.

Heterocycloalkyl: The term “heterocycloalkyl” means a non-aromatic monocyclic or polycyclic ring comprising carbon and hydrogen atoms and at least one heteroatom, preferably, 1 to 4 heteroatoms selected from nitrogen, oxygen, and sulfur. A heterocycloalkyl group can have one or more carbon-carbon double bonds or carbon-heteroatoms double bonds in the ring as long as the ring is not rendered aromatic by their presence. Examples of heterocycloalkyl groups include aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 2 to 6 carbon atoms and from 1 to 3 heteroatoms, referred to herein as (C1-C6)heterocycloalkyl.

Hydrate: As used herein, the term “hydrate” refers to a form of a compound (or of a pharmaceutically acceptable salt thereof) that includes a stoichiometric or non-stoichiometric amount of water, typically retained by non-covalent intermolecular forces. In many embodiments, a solvate form is a solid form; in some embodiments, a solvate form is a crystal form.

Halogen: The term “halogen” means fluorine, chlorine, bromine, or iodine. Correspondingly, the term “halo” means fluoro, chloro, bromo, and iodo.

Carbomer: As used herein, “carbomer” is the USP designation for various polymeric acids that are dispersible but insoluble in water. When the acid dispersion is neutralized with a base a clear, stable gel is formed.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Solvate: As used herein, the term “solvate” refers to a form of a compound (or of a pharmaceutically acceptable salt thereof) that includes a stoichiometric or non-stoichiometric amount of solvent, typically retained by non-covalent intermolecular forces. In many embodiments, a solvate form is a solid form; in some embodiments, a solvate form is a crystal form.

### DETAILED DESCRIPTION OF CERTAIN EMBODIMENTS

The underlying causes of many activated skin disorders are unclear or unknown. Many current treatments target immune or inflammatory responses, but these treatments may have unreliable efficacy and/or undesired side-effects in some patients, thus new treatment options and methods are needed. The present disclosure provides the insight that certain activated skin disorders may be amenable to therapy that targets energy production, and particularly to therapy comprising dermal administration of therapy.

### Activated Skin Disorders

A number of skin disorders are associated with increased cell activation and/or proliferation. Activated or proliferating cells do not necessarily have to be skin or mucous membrane cells but can involve other tissues, such as, for example, cells of the immune system. For purposes of the present disclosure, the term “activated skin disorder”, in some embodiments, may be used to refer to one or more diseases, disorders, or conditions associated with cell activation and/or with cell proliferation. In some embodiments, the term may be used specifically to refer to diseases, disorders or conditions associated with cell activation and/or proliferation. In some embodiments, a skin disorder to which teachings of the present disclosure apply may be, for example, psoriasis, warts, including genital warts, hyperkeratosis, ichthyosis, keratoderma, lichen planus, eczema or atopic dermatitis, and/or cervical dysplasia.

Psoriasis

In some embodiments, an activated skin disorder is psoriasis. The cause of psoriasis is not completely understood. A diagnosis of psoriasis is usually based on the appearance of the skin. Psoriasis shows marked signs of inflammation with pronounced infiltration of leukocytes and lymphocytes. A mixed leukocytic infiltrate is seen in both dermis and epidermis. As a histopathological hallmark of psoriatic lesions, neutrophil granulocytes migrate through the epidermis and form the telltale Munro micro abscesses underneath the stratum corneum. As the lesions progress these micro abscesses are transported to the upper layers of the stratum corneum where they slough off. There are no special blood tests or diagnostic procedures for psoriasis. Psoriasis is not a disease that occurs naturally in animals, which has hindered research of this disease. Psoriasis can occur at any age, though many cases first appear before 35 years of age. Psoriasis includes plaque psoriasis, flexural psoriasis, seborrheic psoriasis, guttate psoriasis, pustular psoriasis, as well as localized forms.

In some embodiments, an activated skin disorder is plaque psoriasis. Plaque psoriasis is the most common form and is characterized by well delineated red, scaly patches on the skin. The scaly patches caused by psoriasis, called psoriatic plaques, are areas of inflammation and excessive skin production. Plaques frequently occur on the skin of the elbows and knees, but can affect any area including the scalp and genitals. In contrast to eczema, psoriasis is more likely to be found on the extensor aspect of the joint. Plaque formation is variable, ranging from one or more localized patches to complete body coverage. In some embodiments, an activated skin disorder is flexural psoriasis. Flexural psoriasis (also known as inverse or intertriginous) refers to plaque psoriasis at submammary, groin, axillary, genital and natal cleft sites, and is typically less scaly. In some embodiments, an activated skin disorder is seborrhoeic psoriasis (“sebopsoriasis”). Sebopsoriasis is similar in appearance and distribution to seborrhoeic dermatitis (hence the name) and may occur in isolation or associated with plaque psoriasis elsewhere. In some embodiments, an activated skin disorder is guttate psoriasis. Guttate psoriasis is characterized by an acute eruption of small (<1 cm) papules of psoriasis which appear over a period of a month or so, and in some subjects is preceded by a streptococcal infection. In some embodiments, an activated skin disorder is pustular psoriasis, including generalized pustular psoriasis (GPP) as well as localized forms (e.g., palmoplantar pustulosis and acrodermatitis continua of Halopeau). Occasionally combinations of the different types develop simultaneously or sequentially over time in the same person. In some embodiments, an activated skin disorder is one or more of plaque psoriasis, flexural psoriasis, seborrheic psoriasis, guttate psoriasis, pustular psoriasis. In some cases, psoriasis follows a relapsing and remitting course in a subject.

The most widely accepted pre-clinical model for psoriasis is in a severe combined immunodeficiency (“SCID”) mouse that lack major elements of a normal immune system. In the absence of a functioning immune system it is possible to transplant human psoriatic skin onto SCID mice. Skin can be maintained in these mice for weeks and investigational drugs can be studied. In a recent review of data using this model it was stated that “This model demonstrated conclusively that psoriasis is a T-cell-mediated disease.” (Gudjonsson, et. al., Journal of Investigative Dermatology (2007) 127, 1292-1308). This view is consistent with the fact that clinically effective treatments of psoriasis are generally anti-inflammatory or immunosuppressive in nature. It is therefore surprising, that in at least some embodiments, an agent that targets energy metabolism (e.g. an anti-glycolytic agent), can be used as an effective treatment for psoriasis.

Warts

In some embodiments, an activated skin disorder is warts. Warts are small, self-limited tumors that may be caused by human papillomavirus. In some embodiments, warts include common warts, flat warts, plantar warts, periungual warts, filiform warts and genital warts.

Hyperkeratosis

In some embodiments, an activated skin disorder is hyperkeratosis. Hyperkeratosis is characterized by the thickening of the outermost layer of the epidermis and is often associated with the presence of an abnormal quantity of keratin. Hyperkeratosis includes follicular hyperkeratosis, plantar hyperkeratosis, hyperkeratosis of the nipple and areola, hyperkeratosis lenticularis perstans, epidermolytic hyperkeratosis, multiple minute digitate hyperkeratosis, focal acral hyperkeratosis and keratosis pilaris.

Ichthyosis

In some embodiments, an activated skin disorder is ichthyosis. Ichthyosis is a heterogeneous family of at least 28 skin disorders characterized by dry, thickened, scaly and/or flaky skin. Skin disorders under the umbrella of ichthyosis include Ichthyosis vulgaris, Ichthyosis acquisita, X-linked ichthyosis, congenital ichthyosiform erythroderma nonbullous (nbCIE), epidermolytic hyperkeratosis, harlequin-type ichthyosis, Ichthyosis bullosa of Siemens, Ichthyosis hystrix, Lamellar ichthyosis types 1-5, CHILD syndrome, Conradi-Hünermann syndrome, Ichthyosis follicularis with alopecia and photophobia syndrome, Keratitis-ichthyosis-deafness syndrome, Netherton syndrome, Sjögren-Larsson syndrome, Photosensitive trichothiodystrophy (IBIDS syndrome) and Gaucher disease.

Keratoderma

In some embodiments, an activated skin disorder is keratoderma. Keratoderma is characterized by marked thickening of the skin. Keratoderma includes simple, complex and acquired keratodermas, such as palmoplantar keratoderma, mal de Meleda, focal palmoplantar keratoderma, striate palmoplantar keratoderma, punctate palmoplantar keratoderma, spiny keratoderma, focal acral hyperkeratosis, erythrokeratodermia variabilis, palmoplantar keratoderma of Sybert, Olmsted syndrome, Naegeli-Franceschetti-Jadassohn syndrome, Papillon-Lefevre syndrome, Pachyonychia congenita types I and II, focal palmoplantar keratoderma with oral mucosal hyperkeratosis, Camisa disease, ectodermal dysplasias, Clouston's hidrotic ectodermal dysplasia, Acrokeratotic poikiloderma, Dermatopathic pigmentosa reticularis, syndromic keratodermas, Vohwinkel syndrome, Naxos disease, keratitis-ichthyosis-deafness syndrome, Corneodermatosseous syndrome, Huriez syndrome, Oculocutaneous tyrosinemia, Cardiofaciocutaneous syndrome, Schopf-Schulz-Passarge syndrome, AIDS-associated keratoderma, arsenical keratosis, calluses, climacteric keratoderma, clavi (corns), eczema, Norwegian scabies, psoriasis, lichen planus, and drug-induced keratoderma.

Lichen Planus

In some embodiments, an activated skin disorder is Lichen planus. Lichen planus is a disease of the skin and/or mucous membranes that is characterized by a resemblance to lichen. The cause of lichen planus is unknown, but is thought to be the result of an autoimmune process with an unknown initial trigger. There is currently no cure for Lichen planus. The lesions associated with lichen planus can occur in many different forms including annular, linear, hypertrophic, atrophic, bullous, ulcerative and pigmented. One common presentation of lichen planus is as a well-defined area of purple-colored, itchy, flat-topped papules with interspersed lacy white lines (Wickham's striae).

Eczema

In some embodiments, an activated skin disorder is eczema or atopic dermatitis. In some cases, the terms “eczema” and “atopic dermatitis” are use synonymously. Eczema describes a range of persistent skin conditions that may be characterized by one or more of the following symptoms: redness, skin swelling, itching and dryness, crusting, flaking, blistering, cracking, oozing, or bleeding. The cause of eczema is unclear.

Cervical Dysplasia

In some embodiments, an activated skin disorder is cervical dysplasia. Cervical dysplasia is characterized by abnormal cell growth on the surface lining of the cervix or endocervical canal in a subject.

### Current Treatments for Skin Activated Disorders

Immunosuppressive drugs have been used in the treatment of activated skin disorders such as psoriasis, e.g., cyclosporine A, methotrexate, infliximab (Remicade®), adalimumab (Humira®) or etanercept (Enbrel®). These potent immunosuppressive drugs are also used in the treatment of organ transplant patients, to prevent the rejection of the foreign organ, or used to treat other auto-immune diseases such as rheumatoid arthritis and are associated with a number of side effects including increased risk for infection. There are also recently developed biological drugs that interact specifically with receptors and signaling compounds of the immune system such as tumor necrosis factor alpha (TNFα). These drugs are very effective, generally well tolerated, but have a number of disadvantages. Biological drugs can have an immune-suppressive effect, diminishing the body's ability, e.g., to fight infections. Also, biological drugs are extremely expensive and, therefore, only appropriate for the most severe cases of activated skin disorders, e.g., psoriasis. Further, due to their biological nature, they cannot be applied directly to the skin or taken by mouth but must be injected.

Steroids are a widely prescribed topical treatment for activated skin disorders such as psoriasis or eczema. However, steroids are burdened with a number of side effects. If used in higher doses or over prolonged periods of time, steroids can enter the bloodstream and lead to reversible Hypothalamic-Pituitary-Adrenal (HPA) axis suppression with the potential for gluco-corticosteroid insufficiency after withdrawal from treatment. Manifestations of Cushing syndrome, hyperglycemia, and glucosuria can also occur in some patients by systemic absorption of topical corticosteroids while on therapy. At the site of skin application, steroids may lead to dryness, hypertrichosis, acneiform eruptions, hypopigmentation, allergic contact dermatitis, secondary infections, irritation, striae, and miliaria. Some of these adverse events may be irreversible.

Vitamin A derivatives such as retinoids (e.g. Tazorac®) and vitamin D derivatives such as calcipotriene (Dovonex®) have also been used to treat activated skin disorders such as psoriasis. Vitamin D and its analogues, such as calcipotriene, can be effective at higher doses. However, it was shown that vitamin D also impacts calcium homeostasis and its use has, therefore, been restricted to a sub optimal dose of <100 g per week. In addition, a significant number of patients experience local side effects such as skin irritation, which is also a frequent side effect of vitamin A and its derivatives.

Other topical drugs such as dithranol (anthralene) or coal tar have unreliable efficacy and are limited by practical aspects such as strong odor or their potential to permanently stain clothing. Hence, new and better treatments for activated skin disorders such as psoriasis are needed.

Certain activated skin disorders, such as psoriasis, are seen as diseases of the immune system and, as described above, are commonly treated by immuno-suppression or anti-inflammatory drugs. It is therefore unexpected and surprising that now a new mode of treatment of psoriasis has been discovered by the use of compounds that do not target the immune system, but instead interfere with the energy production of cells.

### Energy Regulation

In some embodiments, the present disclosure encompasses the insight that certain agents that can impact energy production and/or cellular metabolism can be uniquely useful in the treatment of certain skin disorders. Without wishing to be bound by any particular theory, the present disclosure provides the insight that impacting ATP production can effectively treat certain skin disorders, such as activated skin disorders (e.g. psoriasis), and further provides the particular insight that certain agents that target energy production, as described herein, may be able to achieve beneficial results while avoiding one or more undesirable or negative attributes of potential alternative agents.

In some embodiments, a therapy for treatment of an activated skin disorder comprising administering one or more compounds that targets production of adenosine triphosphate (ATP). ATP is a coenzyme that transports chemical energy within cells. ATP can be produced by redox reactions using carbohydrates or lipids as an energy source. ATP can be produced in animal cells by glycolysis, oxidative phosphorylation and substrate-level phosphorylation. Glycolysis is the metabolic pathway that converts glucose into pyruvate. Oxidative phosphorylation is the metabolic pathway in which the mitochondria in cells use their structure, enzymes, and energy released by the oxidation of nutrients to form ATP. Substrate-level phosphorylation refers to metabolic reactions that results in the formation of ATP or GTP by the direct transfer and donation of a phosphoryl group to adenosine diphosphate (ADP) or guanosine diphosphate (GDP) from a phosphorylated reactive intermediate.

### Inhibitory Agents

In some embodiments, a therapy that targets energy production may include administration of an agent that impairs or inhibits glycolysis directly or indirectly, an agent that impairs mitochondrial function, a mitochondrial poison, or an agent that otherwise interferes with the energy (ATP) production of a cell (e.g., inhibitors of beta oxidation, inhibitors of nucleoside diphosphate kinases, etc.).

In some embodiments, an agent that targets energy production is represented by the general formula:

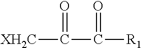

wherein X represents a halide, a sulfonate, a carboxylate, an alkoxide, or an amine oxide. In certain embodiments, X is a halide selected from the group consisting of: fluoride, bromide, chloride, and iodide. In some embodiments, X is a sulfonate. In certain embodiments, a sulfonate is selected from the group consisting of: triflate, mesylate and tosylate. In some embodiments, X is an amine oxide such as dimethylamine oxide. In certain embodiments, R1 represents OR, H, N(R″)2, C1-C6 alkyl, C6-C12 aryl, C1-C6 heteroalkyl, or a C6-C12 heteroaryl. Independently, in other embodiments, R″ represents H, C1-C6 alkyl, or C6-C12 aryl. Independently, in still other embodiments, R represents H, alkali metal, C1-C6 alkyl, C6-C12 aryl or C(O)R′; and R′ represents H, C1-C20 alkyl or C6-C12 aryl. In some embodiments, an agent that targets energy production is a 3-halopyruvate. In certain embodiments, a 3-halopyruvate is selected from the group consisting of: 3-fluoropyruvate, 3-chloropyruvate, 3-bromopyruvate and 3-iodopyruvate. In one embodiment, a 3-halopyruvate is 3-bromopyruvate.

In certain embodiments, an agent that targets energy production is represented by the general formula:

X—CH₂—CO—COOH

wherein X represents a halide, a sulfonate, a carboxylate, an alkoxide, or an amine oxide. In some certain embodiments, X is a halide selected from the group consisting of: fluoride, bromide, chloride, and iodide. In some embodiments, X is a sulfonate is selected from the group consisting of: triflate, mesylate and tosylate. In some certain embodiments, X is an amine oxide such as dimethylamine oxide. In some embodiments, an agent that targets energy production is a 3-halopyruvate. In certain embodiments, a 3-halopyruvate is selected from the group consisting of: 3-fluoropyruvate, 3-chloropyruvate, 3-bromopyruvate and 3-iodopyruvate. In one embodiment, a 3-halopyruvate is 3-bromopyruvate.

Glycolysis Inhibitors

In some embodiments, a therapy for treatment of an activated skin disorder comprising administering an effective amount of an anti-glycolytic agent. In some embodiments, an anti-glycolytic agent is an inhibitor of one or more enzymes in the glycolytic pathway. Glycolysis refers to the series of enzymatic reactions which convert a molecule of glucose into lactate with the generation of two molecules of ATP. The metabolic steps and enzymes involved are well understood in the art (for a detailed description, see, for example, https://en.wikipedia.org/wiki/Glycolysis). Anti-glycolytic agents refer to compounds that impair or inhibit glucose metabolism. In some embodiments, anti-glycolytic agents bind to enzymes involved in the glycolysis pathway and impair or inhibit their activity.

Hexokinase Inhibitors

In some embodiments, an anti-glycolytic agent is a hexokinase inhibitor. Hexokinase catalyzes the first step of glycolysis, phosphorylating hexose (e.g., glucose) to hexose-6-phosphate. Hexokinase inhibitors include 2-deoxyglucose, 5-thioglucose, mannoheptulose, 3-halopyruvate (e.g. 3-bromopyruvate), lonidamine and methyl jasmonate. Analogs, derivatives, prodrugs, or metabolites and salts thereof may also be used, provided they have anti-glycolytic activity. In some embodiments, an anti-glycolytic agent is a 3-halopyruvate. In certain embodiments, an anti-glycolytic agent is selected from the group consisting of: 3-fluoropyruvate, 3-chloropyruvate, 3-bromopyruvate and 3-iodopyruvate. In some embodiments, other enzymes in the glycolytic pathway are inhibited such as for example glyceraldehyde-3-phosphate dehydrogenase (GAPDH).

3-Bromopyruvate

In some embodiments, an anti-glycolytic agent is 3-bromopyruvate. In some embodiments, analogs, derivatives, prodrugs, or metabolites of 3-bromopyruvate and salts thereof may also be used, provided that they have anti-glycolytic activity. Preferably, analogs, derivatives, prodrugs, or metabolites of 3-bromopyruvate and salts thereof have an anti-proliferative effect that is statistically similar to or greater than that of 3-bromopyruvate, e.g., for treating psoriasis by topical administration. When referring herein to a treatment using 3-bromopyruvate, it should be understood that the treatment may also be conducted with analogs, derivatives, prodrugs, metabolites of 3-bromopyruvate and salts thereof, where applicable.

The anti-glycolytic agent 3-bromopyruvate has previously been characterized as an agent for the treatment of cancer. See, for example, U.S. 2010/0203110 and U.S. Pat. No. 7,547,673. However, there was no indication that 3-bromopyruvate would be an effective treatment for other non-malignant disorders, such as activated dermal disorders, that do not require eradication of cells. As described herein, 3-bromopyruvate is a promising therapy for skin disorders associated with activation of cells, such as psoriasis, eczema, warts, etc. In some embodiments, treatment of an activated skin disorder comprises administering 3-bromopyruvate in an amount insufficient to induce cell death or that minimally induces cell death in the target tissue (e.g. cells in skin, which may include activated and/or non-activated cells). In some embodiments, treatment of an activated skin disorder comprises administering 3-bromopyruvate in an amount insufficient to eradicate the activated cells in the target tissue (e.g. cells in skin to which the 3-bromopyruvate is administered). In some embodiments, treatment of an activated skin disorder comprises administering 3-bromopyruvate in an amount insufficient to inhibit cell proliferation or that minimally inhibits cell proliferation in the target tissue (e.g. cells in skin, which may include activated and/or non-activated cells).

Other Anti-Glycolytic Targets

In some embodiments, an anti-glycolytic agent is a glyceraldehyde-3 phosphate dehydrogenase (GAPDH) inhibitor. GAPDH converts glyceraldehyde-3-phosphate to 1,3-bisphospho-glycerate with a simultaneous reduction of NAD⁺ to NADH (nicotinamide adenine dinucleotide; oxidized and reduced respectively). GAPDH inhibitors include, for example, alpha-chlorhydrin, ornidazole, arsenic, iodoacetate, koningic acid, and 3-bromopyruvate. In some embodiments, an anti-glycolytic agent is a lactate dehydrogenase inhibitor. Lactate dehydrogenase catalyzes the conversion of lactate to pyruvate as the last step in anaerobic glycolysis. Lactate dehydrogenase inhibitors include, for example, oxamate and oxalate. In some embodiments, an anti-glycolytic agent is gossypol or a gossypol analog.

Inhibitors of Mitochondrial Function and Mitochondrial Poisons

In some embodiments, a therapy for treatment of an activated skin disorder comprising administering an effective amount of an agent that impairs mitochondrial function. In some embodiments, an agent that impairs mitochondrial function is an ATPase inhibitor. ATPase inhibitors include, for example, apoptolidin. In some embodiments, a therapy for treatment of an activated skin disorder comprising administering an effective amount of a mitochondrial poison. Mitochondrial poisons include, for example, comprises a Complex I inhibitor, a Complex II inhibitor, a Complex III inhibitor, a Complex IV inhibitor, a Complex V inhibitor or an uncoupling agent.

Identification and/or Characterization of Inhibitory Agents

Analogously, in some embodiments, the present disclosure provides methods of identification and/or characterization of agents that target energy production that can be useful in the treatment of activated skin disorders.

In some embodiments, suitable agents that target energy production may be identified and/or characterized using in vitro ATP assays. These assays are commercially available, which are well known in the art. Examples of in vitro ATP assays include, the Molecular Probes™ (Eugene, Oreg.) ATP Determination Kit and the Sigma-Aldrich (St. Louis, Mo.) Adenosine 5′-triphosphate (ATP) Bioluminescent Assay Kit. In some embodiments, suitable agents of the present disclosure may be identified and/or characterized for their ability to inhibit cell proliferation in vitro or in vivo. Assays to assess in vivo or in vitro cell proliferation are well known in the art. Assays to assess cell proliferation include, but are not limited to, commercially available kits, such as the series of Click-iT Plus EdU Imaging Kits (ThermoFisher Scientific, Cambridge, Mass.).

In some embodiments, suitable agents of the present disclosure may be identified and/or characterized for their ability to induce cell death in vitro or in vivo. Assays to assess cell death and/or cell viability are well known in the art. Assays to assess cell death include, but are not limited to, commercially available kits. Examples of assays to assess cell death or cell viability include, but are not limited to, assays that employ dye uptake/exclusion, for example SYTOX® dead cell stains and LIVE/DEAD® Imaging kits, both available from ThermoFisher Scientific. Assessing cell death or cell viability may involve quantifying the “counts per microscopic field”, microplate assays, and/or flow cytometry analysis, etc.

In some embodiments, suitable agents of the present disclosure may be identified and/or characterized for their ability to induce apoptosis or programmed cell death in vivo or in vitro. Assays to assess apoptosis and/or programmed cell death are well known in the art and include, but are not limited to, commercially available kits.

In some embodiments, suitable agents of the present disclosure may be identified and/or characterized by assays to detect oxidative stress or damage to cells in vivo or in vitro. Assays to detect oxidation and/or oxidation induced damage to cells in vivo or in vitro are well known in the art. In vivo and in vitro assays to detect oxidation and/or oxidation induced damage to cells include, but are not limited to, commercially available kits.

In some embodiments, suitable agents of the present disclosure may be identified and/or characterized by assays to assess in vivo or in vitro cellular cytotoxicity, mitotoxicity and/or genotoxicity. In some embodiments, assays to assess cellular cytotoxicity, mitotoxicity and/or genotoxicity are well known in the art and include, but are not limited to, commercially available kits.

### Dosage

In some embodiments, dosages and dosing frequency will be determined by a trained medical professional depending on the activity of the compound of the present disclosure, the characteristics of the particular topical formulation, and the identity and severity of the dermatologic disorder treated or prevented. In general, an agent that targets energy production is present in a formulation of the present disclosure in an amount of from about 0.0001% to about 50% of the total weight of the formulation, or from about 0.005% to about 25%, or from about 0.01% to about 10% of the total weight of the formulation. In some embodiments, an agent that targets energy production is present in the topical formation as, for example, at 20%, 2%, 0.2%, or 0.02% of the total weight of the formulation. In some embodiments, an agent that targets energy production may be present in a topical formulation in an amount below about 25%; in some embodiments, an agent that targets energy production may be present in a topical formulation in an amount above about 0.01%, 0.02%, 0.03%, 0.04%, 0.05% or more; in some embodiments above about 0.1%, 0.2%, 0.3%, 0.4%, 0.5% or more; in some embodiments about 1%, 2%, 3%, 4%, 5%, or more. In some embodiments, an agent that targets energy production may be present in a topical formulation in an amount within a range of about 0.01% to about 50%, 0.1% to about 40%, 0.02% to about 30%, 0.03% to about 25%; in some embodiments within a range of about 0.05% and about 5%; in some embodiments within a range of about 1% to about 20%, etc.

In some embodiments, a formulation comprises an anti-glycolytic agent that is present in a formulation of the present disclosure in an amount from about 0.0001% to about 50% of the total weight of the formulation, or from about 0.005% to about 25%, or from about 0.01% to about 10% of the total weight of the formulation. In some embodiments, an anti-glycolytic agent is present in the topical formation as, for example, at 20%, 2%, 0.2%, or 0.02% of the total weight of the formulation. In some embodiments, an anti-glycolytic agent may be present in a topical formulation in an amount below about 25%; in some embodiments, an anti-glycolytic agent may be present in a topical formulation in an amount above about 0.01%, 0.02%, 0.03%, 0.04%, 0.05% or more; in some embodiments above about 0.1%, 0.2%, 0.3%, 0.4%, 0.5% or more; in some embodiments about 1%, 2%, 3%, 4%, 5%, or more. In some embodiments, an anti-glycolytic agent may be present in a topical formulation in an amount within a range of about 0.01% to about 50%, 0.1% to about 40%, 0.02% to about 30%, 0.03% to about 25%; in some embodiments within a range of about 0.05% and about 5%; in some embodiments within a range of about 1% to about 20%, etc.

In some embodiments, a formulation comprises 3-bromopyruvate or an analog, derivative, prodrug, or metabolite or salt thereof, that is present in a formulation in an amount from about 0.0001% to about 50% of the total weight of the formulation, or from about 0.005% to about 25%, or from about 0.01% to about 10% of the total weight of the formulation.

The present disclosure provides, among other things, a surprising recognition that non-toxic or minimally toxic levels of agents that target energy production (e.g., an anti-glycolytic agent) may be effective to treat certain activated skin disorders. In some embodiments, an agent that targets energy production (e.g., an anti-glycolytic agent), is administered in an amount insufficient to kill cells of the target tissue (e.g. cells found in skin, which may include activated and/or non-activated cells). In some embodiments, a therapy for treatment of an activated skin disorder comprises administering an effective amount of an agent that targets energy production (e.g., an anti-glycolytic agent), wherein the agent induces little or no cell death in the non-activated cells of a target tissue. In some embodiments, a therapy for treatment of an activated skin disorder comprises administering an effective amount of an agent that targets energy production (e.g., an anti-glycolytic agent), wherein the agent induces little or no cell death in the both the activated and non-activated cells of a target tissue. In some embodiments, an agent that targets energy production may be administered in an amount that induces less than 50%, 45%, 40%, 35%, 30%, 25%, 20%, 15%, 10%, 9%, 8%, 7%, 6%, 5%, 4%, 3%, 2%, 1%, 0.5%, 0.1% cell death in an appropriate in vitro assay.

In some embodiments, an agent that targets energy production (e.g., an anti-glycolytic agent), is administered in an amount effective to inhibit cell proliferation. In some embodiments, an agent that targets energy production (e.g., an anti-glycolytic agent), is administered in an amount that is ineffective for inhibiting cell proliferation (e.g., minimal inhibition or no inhibition of cell proliferation).

To treat or prevent recurrence of activated skin conditions, the topical formulations of the present disclosure are topically applied directly to the affected area in any conventional manner well known in the art. For example, by dropper or applicator stick, as a mist via an aerosol applicator, via an intra-dermal or trans-dermal patch or film, or by simply spreading a formulation onto the affected area with fingers. In some embodiments, an amount of a topical formulation applied to an affected skin area ranges from about 0.0005 g/cm² of skin surface area to about 0.5 g/cm², preferably, 0.001 g/cm² to about 0.01 g/cm² of skin surface area. In some embodiments, 1, 2, 3, 4, 5, 6, 7, 8, 9, or 10 applications per day are recommended during treatment. In some embodiments applications are once per day or less frequently, such as every other day or weekly. In some embodiments, one to four applications per day are recommended during the term of treatment.

### Dermal Formulations

Topical Formulations

In one embodiment, the compounds of the present disclosure are delivered to the affected area of the skin in a pharmaceutically acceptable topical carrier. As used herein, a pharmaceutically acceptable topical carrier is any pharmaceutically acceptable formulation that can be applied to the skin or mucosal surface for topical, dermal, intradermal, or transdermal delivery of a pharmaceutical or medicament. The combination of a pharmaceutically acceptable topical carrier and a compound of the present disclosure is termed a topical formulation. Topical formulations of the present disclosure are prepared by mixing a compound of the present disclosure with a topical carrier according to well-known methods in the art, for example, methods provided by standard reference texts such as, REMINGTON: THE SCIENCE AND PRACTICE OF PHARMACY 1577-1591, 1672-1673, 866-885; (Alfonso R. Gennaro ed. 19th ed. 1995); Ghosh, T. K.; et al. TRANSDERMAL AND TOPICAL DRUG DELIVERY SYSTEMS (1997), both of which are hereby incorporated herein by reference.

The topical carriers useful for topical delivery of compounds of the present disclosure can be any carrier known in the art for topically administering pharmaceuticals, for example, but not limited to, pharmaceutically acceptable solvents, such as a polyalcohol or water; emulsions (either oil-in-water or water-in-oil emulsions), such as creams or lotions; micro- or nano-emulsions; gels; ointments; liposomes; powders; and aqueous solutions or suspensions, such as standard ophthalmic preparations. In some embodiments, an agent for the treatment or prevention of activated skin disorder, such as psoriasis, is administered topically as a dermatological product such as gels, creams, ointments, foams, patches, films or others.

In some embodiments, an agent that targets energy production is present in the topical formulation an amount in the range of about 0.0001 percent to about 50 percent of the total weight of the formulation. In some embodiments, an agent that targets energy production is present in the topical formation as 0.02 to 20% of the total weight of the formulation. In some embodiments, the topical formulation has a pH value in the range of about 2.5 to 8 or about 3-6.

Emulsions, Gels, and Ointments as Topical Carriers

In some embodiments, the topical carrier used to deliver a compound of the present disclosure is an emulsion, gel, or ointment. Emulsions, such as creams and lotions are suitable topical formulations for use in the present disclosure. An emulsion is a dispersed system comprising at least two immiscible phases, one phase dispersed in the other as droplets ranging in diameter from 10 nm to 1,000 μm. An emulsifying agent is typically included to improve stability. When water is the dispersed phase and oil is the dispersion medium, the emulsion is termed a water-in-oil emulsion. When oil is dispersed as droplets throughout the aqueous phase as droplets, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In some embodiments, the topical carrier used to deliver a compound of the present disclosure is a gel, for example, a two-phase gel or a single-phase gel. Gels are semi-solid systems consisting of suspensions of small inorganic particles or large organic molecules interpenetrated by a liquid. When the gel mass comprises a network of small discrete inorganic particles, it is classified as a two-phase gel. Single phase gels consist of organic macromolecules distributed uniformly throughout a liquid such that no apparent boundaries exist between the dispersed macromolecules and the liquid. Suitable gels for use in the present disclosure are disclosed in REMINGTON: THE SCIENCE AND PRACTICE OF PHARMACY 1517-1518 (Alfonso R. Gennaro ed. 19th ed. 1995), hereby incorporated herein by reference. Other suitable gels for use with the present disclosure are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002), each of which patents is hereby incorporated herein by reference.

Polymer thickeners (gelling agents) that may be used include those known to one skilled in the art, such as hydrophilic and hydro-alcoholic gelling agents frequently used in the cosmetic and pharmaceutical industries. The hydrophilic or hydro-alcoholic gelling agent may comprise “CARBOPOL®” (B.P. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). In some embodiments, a formulation of the present disclosure comprises a gelling agent as a compositional weight percent between about 0.2% to about 4% by weight of the composition. In some embodiments, the gelling agent comprises 0.1% to 10%, 0.2% to 8%, 0.3% to 6%, 0.4% to 6%, 0.2% to 5%, 0.2% to 2%, 0.5% to 4%, 0.5% to 2% by weight of the composition. A preferred weight percent range for “CARBOPOL®” is between about 0.5% to about 2%, while a preferred weight percent range for “NATROLSOL®” and “KLUCEL®” is between about 0.5% to about 4%. The preferred compositional weight percent range for both “HYPAN®” and “STABILEZE®” is between 0.5% to about 4%.

In some embodiments, a topical formulation of the present disclosure comprises a gelling polymer. Gelling polymers include carbomers. “CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. Other carbomers include 910, 934P, 940, 941, and 1342. “KLUCEL®” is a cellulose polymer that is dispersed in water and forms a uniform gel upon complete hydration. Gelling polymers also include hydroxyl-ethyl-cellulose, cellulose gum, MVE/MA decadiene cross polymer, PVM/MA copolymer, or a combination thereof.

In another embodiment, the topical carrier used to deliver a compound of the present disclosure is an ointment. Ointments are oleaginous semisolids that contain little if any water. Preferably, the ointment is hydrocarbon based, such as a wax, petrolatum, or gelled mineral oil. Suitable ointments for use are well known in the art and are disclosed in REMINGTON: THE SCIENCE AND PRACTICE OF PHARMACY 1585-1591 (Alfonso R. Gennaro ed. 19th ed. 1995), hereby incorporated herein by reference.

In some embodiments, a topical formulation of the present disclosure has a viscosity in a range of ˜10 to 1,250,000 centipoise (cP). In some embodiments, a topical formulation of the present disclosure has a viscosity in a range of ˜10 to 80,000 cP. In some embodiments, a topical formulation of the present disclosure has a viscosity in a range of ˜30,000 to 750,000 cP. In some embodiments, a topical formulation of the present disclosure has a viscosity in a range of ˜30,000 to 300,000 cP. In some embodiments, a topical formulation of the present disclosure has a viscosity in a range of 500,000 to 1,250,000 cP. In some embodiments, a topical formulation of the present disclosure has a viscosity that is greater than 30,000 cP. In some embodiments, a topical formulation of the present disclosure has a viscosity that is less than 500,000 cP. In some embodiments, a topical formulation of the present disclosure has a viscosity that is less than 30,000 cP.

Aqueous Topical Formulations

In another embodiment, a topical carrier used in a topical formulation of the present disclosure is an aqueous solution or suspension, preferably, an aqueous solution. Well-known ophthalmic solutions and suspensions are suitable topical carriers for use in the present disclosure. Suitable aqueous topical formulations for use in the present disclosure are disclosed in REMINGTON: THE SCIENCE AND PRACTICE OF PHARMACY 1563-1576 (Alfonso R. Gennaro ed. 19th ed. 1995), hereby incorporated herein by reference. Other suitable aqueous topical carrier systems are disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); U.S. Pat. No. 6,465,464 (issued Oct. 15, 2002), all of which patents are hereby incorporated herein by reference.

The pH of an aqueous topical formulation of the present disclosure are preferably within the range of about 2.5 to about 8, or about or about pH 3 to 7, or about pH 3 to 6.5, or about pH 3 to 6. To stabilize the pH, preferably, an effective amount of a buffer is included. In one embodiment, a buffering agent is present in an aqueous topical formulation in an amount of from about 0.05 to about 1 weight percent of the formulation. Acids or bases can be used to adjust the pH as needed. Suitable buffering agents are listed below. In some embodiments, a topical formulation has a pH value in the range of about pH 2.5 to 8 or about pH 3 to 7, or about pH 3 to 6.

Pharmaceutical Additives

Topical formulations of the present disclosure can include pharmaceuticals or their pharmaceutically acceptable salts, for example, but not limited to, topical corticosteroids and other anti-inflammatory agents, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and antiinfectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

Tonicity-adjusting agents can be included in the aqueous topical formulations of the present disclosure. Examples of suitable tonicity-adjusting agents include, but are not limited to, sodium chloride, potassium chloride, mannitol, dextrose, glycerin, and propylene glycol. The amount of a tonicity agent can vary widely depending on a formulation's desired properties. In some embodiments, a tonicity-adjusting agent is present in a topic formulation in an amount of about 0.1 wt % to about 10 wt %, about 0.2 wt % to about 5 wt %, about 0.3 wt % to about 3 wt %, about 0.4 wt % to about 2 wt %, about 0.5 wt % to about 1 wt %, or about 0.5 wt % to about 5 wt %. In one embodiment, a tonicity-adjusting agent is present in an aqueous topical formulation in an amount of about 0.5 to about 0.9 weight percent of the formulation.

In some embodiments, an aqueous topical formulation of the present disclosure has a viscosity in a range of about 0.5 cps to about 250,000 cps at 20 degrees Celcius. The viscosity of aqueous solutions of the present disclosure can be adjusted by adding viscosity adjusting agents, for example, but not limited to, polyvinyl alcohol, povidone, hydroxylpropyl methyl cellulose, poloxamers, carboxymethylcellulose, or hydroxyethyl cellulose. In some embodiments, an aqueous topical formulation has a viscosity in a range of about 0.5 cps to about 250,000 cps; or about 1 cps to about 10,000 cps; or about 100 cps to about 50,000 cps; or about 1,000 cps to about 200,000 cps; or about 2,000 cps to about 20,000 cps.

In some embodiments, an aqueous topical formulation comprises isotonic saline, a preservative, such as benzalkonium chloride or chlorinedioxide, a viscosity-adjusting agent, such as polyvinyl alcohol, and a buffer system such as sodium citrate and citric acid.

Excipients

Topical formulations of the present disclosure can comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), hereby incorporated herein by reference, including, but not limited to, protectives, adsorbents, demulcents, emollients, preservatives, antioxidants, moisturizers (humectants), buffering agents, solubilizing agents, skin-penetration agents, and surfactants.

Suitable protectives and adsorbents include, but are not limited to, dusting powders, stearates, collodion, dimethicone, silicones, carbonates, aloe vera gel and other aloe products, vitamin E oil, allatoin, glycerin, petrolatum, and zinc oxide.

Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid, and other anti-microbial agents such as chlorhexidine, chlorocresol, benzoic acid and polymyxin.

In some embodiments, topical formulations of the present disclosure comprise chlorine dioxide (ClO₂) or stabilized chlorine dioxide as a preservative. The term “stabilized chlorine dioxide” is well known in the industry and by those skilled in the art. Stabilized chlorine dioxide includes one or more chlorine dioxide precursors such as one or more chlorine dioxide-containing complexes and/or one or more chlorite-containing components and/or one or more other entities capable of decomposing or being decomposed in an aqueous medium to form chlorine dioxide. U.S. Pat. No. 5,424,078 (issued Jun. 13, 1995), hereby incorporated herein by reference, discloses a form of stabilized chlorine dioxide and a method for producing same, which can be used as a preservative for aqueous ophthalmic solutions and is useful in topical formulations of the present disclosure. The manufacture or production of certain stabilized chlorine dioxide products is described in U.S. Pat. No. 3,278,447, hereby incorporated herein by reference. A commercially available stabilized chlorine dioxide which can be utilized in the practice of the present disclosure is the proprietary stabilized chlorine dioxide of BioCide International, Inc. of Norman, Okla., sold under the trademark Purogene™ or Purite™. Other suitable stabilized chlorine dioxide products include that sold under the trademark DuraKlor by Rio Linda Chemical Company, Inc., and that sold under the trademark Antheium Dioxide by International Dioxide, Inc.

Suitable antioxidants include, but are not limited to, ascorbic acid and its esters, sodium bisulfate, butylated hydroxytoluene, butylated hydroxyanisole, tocopherols, and chelating agents like EDTA and citric acid.

Moisturizers (humectants) are compounds for topical administration that prevent loss of moisture, thereby retaining the skin's natural moisture. Some skin humectant compounds also have the ability to actively attract moisture. Suitable moisturizers include, but are not limited to, glycerin, sorbitol, polyethylene glycols, urea, and propylene glycol.

Suitable buffering agents can include, but are not limited to, acetate buffers, citrate buffers, phosphate buffers, lactic acid buffers, and borate buffers.

Suitable solubilizing agents include, but are not limited to, quaternary ammonium chlorides, cyclodextrins, benzyl benzoate, lecithin, and polysorbates.

Suitable skin-penetration agents include, but are not limited to, ethyl alcohol, isopropyl alcohol, octyl-phenyl-polyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Pharmaceutical Additives

The topical formulations of the present disclosure can include pharmaceuticals or their pharmaceutically acceptable salts, for example, but not limited to, topical corticosteroids and other anti-inflammatory agents, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and antiinfectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

Combination Therapy

Formulations as described herein can be used in combination with other treatments and/or medications, for example to provide more effective treatment or prevention recurrence of activated skin conditions and its symptoms. In some embodiments, a topical formulation is used in combination with another treatment regimen and/or medication well known for treatment of dermatologic disorders, such as those disclosed in THE MERCK

MANUAL 811-830 (Keryn A. G. Lane et al. eds. 17th ed. 2001), hereby incorporated herein by reference.

Using a formulation or compound of the present disclosure in combination with another medicament or treatment means administering a compound of the present disclosure and another medicament or treatment to a subject in a sequence and within a time interval such that they can act together to treat, or prevent recurrence of, activated skin conditions, such as psoriasis, and its symptoms. For example, compounds of the present disclosure can be administered at the same time as another medicament, in the same or a separate formulation as another medicament, or administered at different times.

Any suitable route of administration can be employed to deliver an additional treatment or medication including, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation. Thus, the formulations of the present disclosure can be administered together or at separate times with other medications or treatments.

In some embodiments, topical formulations are used in combination with systemic administration of an antibiotic or retinoid including, but not limited to, orally dosed antibiotics, such as clindamycin, tetracycline, minocin, minocycline, erythromycin, and doxycycline, and orally dosed retinoids such as isotretinoins (e.g., Accutane or Roaccutane).

In some embodiments, topical formulations are used in combination with other topical treatments including, but not limited to, topical formulations consisting of salicylic acid, “sal acid”, tar, topical shampoo and bath solutions, moisturizers, anthralin, Vitamin D analogues such as calcipotriene, becocalcidiol, Taclonex®; Vitamin A and its derivatives such as tazarotene; steroids; biological drugs such as Amevive®, Enbrel®, Humira®, Raptiva®, or Remicade®.

In some embodiments, topical formulations are used in combination with radiation therapy such as UVB, UVA, mixed light pulse therapy (photoderm), and/or pulsed dye laser treatment with or without the addition of a chemotherapeutic drug and/or a photosensitizing compound such as psoralen.

### Article of Manufacture

In some embodiments, the present disclosure provides methods of manufacturing a medicament for topical delivery of an agent that targets energy production. In some embodiments, an article of manufacture comprises a topical formulation of the present disclosure in a suitable container with labeling and instructions for use. The container can be a dropper or tube with a suitable small orifice size, such as an extended tip tube made of any pharmaceutically suitable material.

In some embodiments, topical formulations can be filled and packaged into a plastic squeeze bottle or tube. Suitable container-closure systems for packaging topical formulations are commercially available for example, from Wheaton Plastic Products, 1101 Wheaton Avenue, Millville, N.J. 08332.

In some embodiments, instructions are packaged with the formulations of the present disclosure, for example, a pamphlet or package label. The labeling instructions explain how to administer topical formulations of the present disclosure, in an amount and for a period of time sufficient to treat or prevent recurrence of activated skin disorders such as psoriasis and its symptoms. Before a composition can be approved for any particular use in the United States, it must first be approved for marketing by the United States Food and Drug Administration. Part of that process includes providing a label that will accompany the pharmaceutical composition that is ultimately sold. Preferably, a label includes dosage and administration instructions for a topical formulation that is ultimately sold. Preferably, a label includes dosage and administration instructions, a composition of a topical formulation, a description of the clinical pharmacology, drug resistance, pharmacokinetics, absorption, bioavailability, and/or contraindications associated with a composition of the present disclosure.

### EXEMPLIFICATION

### Example 1

The present Example describes topical formulations of 3-bromopyruvate (“3-BP”). Topical formulations within the scope of the present disclosure can include 3-BP and a commercially available cream “compounding base.” Exemplary commercially available compounding bases include, but are not limited to, Vanicream™ Skin Cream products (Pharmaceutical Specialties, Inc.), Freedom's Compounding Bases (Freedom Pharmaceuticals), and Eucerin cream bases (Beiersdorf Inc.). Topical formulations of 3-BP may be generated by mixing a solution of 3-BP with a commercially available cream compounding base to achieve a desired final concentration (e.g., 0.001% w/w, 0.0025% w/w, 0.005% w/w, 0.01% w/w, 0.05% w/w, 0.1% w/w, 0.5% w/w, 1% w/w, 5% w/w, 10% w/w, etc.).

### Example 2

The present Example describes use of an exemplary topical formulation of 3-BP in the treatment of psoriasis. The goal of this study is to reduce the hyper-proliferation of skin cells and deactivate the inflammatory response associated with the condition. Therefore, treatment was planned in a sequential dose escalating fashion. Dose escalation is accomplished by a step wise increase in formulation strength and/or application volume and frequency. Daily or twice daily doses were administered, with an initial strength ranging from about 0.001% to about 0.05% and administered doses were increased incrementally to a final strength ranging from about 5% to about 25% of the total weight of the formulation. As a first objective, the maximum tolerated dose was established, which was indicated by the observation of local irritation in the area of application. In some cases, once local irritation was observed, dosing was then reduced by approximately factor of four and continued or readjusted as appropriate to assess efficacy.

An exemplary topical formulation of 3-BP was prepared as described below:

An exemplary 3-Bromo-Pyruvic Acid (“BPA”) formulation was compounded as a lotion of low viscosity, filled dropper bottles (e.g., a 3 mL dropper bottle).

Compounding: 3-Bromo-Pyruvic Acid, BPA, CAS#1113-59-3, was obtained from AK Scientific, Inc., Union City, Calif. and stored at frozen conditions. Given the known instability of BPA at higher pH (e.g., physiologic pH) the final formulation consisted of 75% acetic acid (5% w/w) and 25% compounding base with a pH of approximately 3.0. During formulation, all ingredients were chilled to +5° C., and kept on ice for the entire procedure.

Stock solution A was prepared by dissolving the appropriate amount of solid bromopyruvic acid, BPA, in 5% acetic acid at a concentration of 2% to 10% (w/w); depending on the desired final concentration, intermediate dilutions of for example 1:5, using 5% acetic acid, may have been prepared as well.

Stock solution B consisted of a commercially available compounding base, Vanicream™, NDC 45334-300-16, that was diluted 1:1 in 5% acetic acid. The final formulation was comprised of blending BPA solution (Stock A) with compounding base (Stock B) at a ratio of 1:1. The resulting formulation is of low viscosity and filled in 3 ml ophthalmic dropper bottles.

Final concentration of BPA was between 0.15 mM (0.0025% w/w) and 300 mM (5.0% w/w). To preserve stability of the active ingredient, the formulation was stored frozen at −18° C. until use. Once treatment application commenced, bottles were kept at refrigerated conditions (+5° C.) for up to 1 week and then replaced with freshly made product.

This research was conducted as an out-patient, open label, intra-individually controlled repeated measurement experiment involving up to 10 subjects. To minimize bias, subjects were treated at selected target plaques. Neighboring plaques serve as an intra-individual control.

An exemplary 3-BP formulation was self-administered as one or two drops per application, depending on size of the treatment area. Care was taken for drops to fall freely from the dropper tip to ensure a consistent dose volume of ≈25-50 μl/drop. Most treatment applications were done in the morning and photographs were taken mostly in the evening to allow possible moisturizing effects of the compounding base to wear off.

Within each treatment area, the severity is estimated by three clinical signs: erythema (redness), induration (thickness) and desquamation (scaling). Efficacy is assessed using the Investigators Global Assessment scale and the Psoriasis Area Severity Index (PASI), which are summarized in Tables 1 and 2 below. In addition, target lesions are photographed and adjudicated by a blinded independent investigator. These efficacy variables were assessed at every visit.

TABLE 1
Investigator's Global Assessment (IGA) of Disease Severity

| Score | Grade | Definition |
|---|---|---|
| 0 | None | No plaque elevation above normal skin level; may have residual non- |
| | | erythematous discoloration; no psoriatic scale; no erythema |
| 1 | Minimal | Essentially flat with possible trace elevation; faint erythema; no |
| | | psoriatic scale |
| 2 | Mild | Slight but definite elevation of plaque above normal skin level; may |
| | | have up to moderate erythema (red coloration); fine scales with some |
| | | lesions partially covered |
| 3 | Moderate | Moderate elevation with rounded or sloped edges to plaque; moderate |
| | | erythema (red coloration); somewhat coarse scales with most lesions |
| | | partially covered |
| 4 | Severe | Marked elevation with hard, sharp edges to plaque; severe erythema |
| | | (very red coloration); coarse, thick scales with virtually all lesions |
| | | covered and a rough surface |
| 5 | Very Severe | Very marked elevation with very hard, sharp edges to plaque; very |
| | | severe erythema (extreme red coloration); very coarse, thick scales |
| | | with all lesions covered and a very rough surface |

Source: From FDA Draft Guidance on Tazarotene, recommended April 2011, http://www.fda.gov/downloads/drugs/guidancecomplianceregulatoryinformation/guidances/ucm283489.pdf

TABLE 2
Psoriasis Area Severity Index (PASI) at the Target Lesion Site

| Score | Grade | Erythema | Scaling | Plaque Elevation |
|---|---|---|---|---|
| 0 | Clear | No evidence of | No evidence of scaling | No evidence of plaques |
| | | erythema | | above normal skin level |
| 1 | Almost | Pink discoloration, | Occasional fine scales | Slight, just discernable |
| | Clear | minimal erythema | hardly noticeable | elevation above normal |
| | | | | skin level |
| 2 | Mild | Light red coloration | Slight but definite | Discernable elevation |
| | | | roughness, fine scale | above normal skin level |
| | | | present, no cracking | upon examination, but |
| | | | | not pronounced |
| 3 | Moderate | Moderate redness, | Moderate roughness, | Definite plaque |
| | | but not dark | somewhat coarse scaling | formation with |
| | | | | rounded/sloped edges to |
| | | | | plaque |
| 4 | Severe | Dark red coloration | Marked roughness, | Marked elevation with |
| | | | coarse/thick scaling, | hard, distinct edges to |
| | | | cracking may be evident | plaque |
| 5 | Very | Very dark red | Very thick scales | Very marked elevation, |
| | Severe | coloration with | covering extensive area | very hard and sharp |
| | | induration present | severe cracking/fissures | edges to plaque |
| | | | may be evident | |

Safety is assessed by collection of research events and review of concomitant treatments. Clinical adverse event reporting is conducted at each visit. Vital signs including weight, height, temperature, resting pulse, blood pressure, and respiration rate are recorded.

Described below is an exemplary case report of subject whose psoriatic plaques were treated with an exemplary 3-BP formulation as described above. The subject, a 58 year old female, was treated topically on her forearms and/or elbows. Treatment of different plaques was initiated sequentially but some overlap in treatment periods occurred.

Plaque 1, Left Forearm

At the commencement of the study, the subject had two psoriatic plaques on her left forearm. One drop (approx. 0.025 ml) of an exemplary 3-BP formulation was applied topically, twice per day. During the first week, dosing was escalated by using formulations of increasingly higher strengths ranging from 0.1%, 0.2%, 0.4%, 0.7%, 1.4%, 2.6% to 5.0%, (6-300 mM), respectively, and was then maintained at that level.

Treatment with the exemplary formulation resulted in a rapid, visible improvement of psoriatic plaques. During the first week, the treated plaque(s) became smoother and flatter and eventually, the plaques completely resolved.

However, local irritation, oozing, and crusting was also observed after approximately one week of treatment. Consequently, dosing frequency was reduced from BID to QD and, subsequently, the strength of the formulation was successively reduced back to the 0.1% (6 mM) level. Treatment was stopped entirely after 6 weeks. Irritation and crusting resolved without sequelae over the next few weeks.

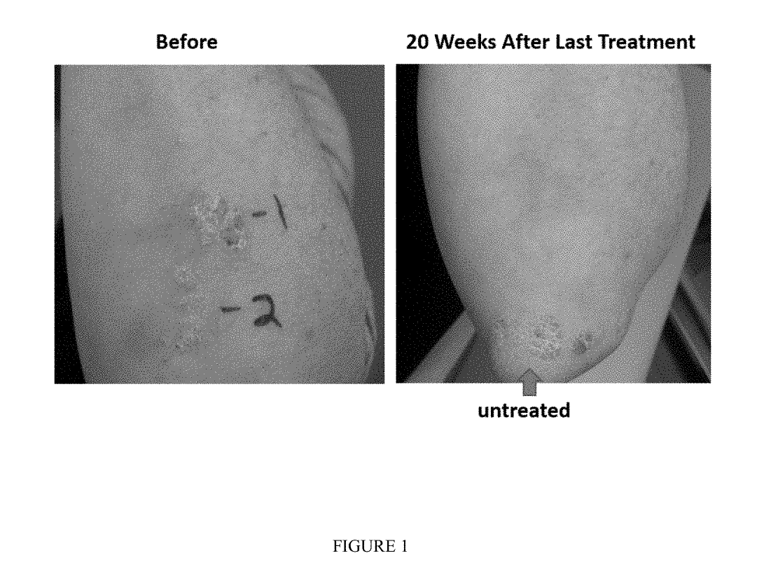

Pictures taken before the start of treatment and 26 weeks after baseline, i.e. 20 weeks after last treatment application, are shown in FIG. 1. Shown is the left forearm of a subject, “before” and 20 weeks “after” last treatment. FIG. 1 illustrates that plaques remain in complete remission even 20 weeks after last treatment application. As of the date of the last visit, the remission has been maintained for more than 8 months after last treatment application. At Week 32, i.e. 26 weeks after the last treatment application, a small plaque evolved adjacent to the previously treated area. Thus, this demonstrates the unexpected ability of an exemplary formulation of 3-BP to successfully resolve a psoriatic plaque, such that even after more than 8 months completion of treatment with an exemplary formulation, these original plaques remain in remission.

Plaque #2, Right Forearm

The same subject received once daily topical applications of an exemplary formulation of 3-BP to a plaque on her right forearm at a concentration of 0.1% (w/w) (6 mM) for one week and at a concentration of 0.03% (w/w) (1.75 mM) for another two weeks.

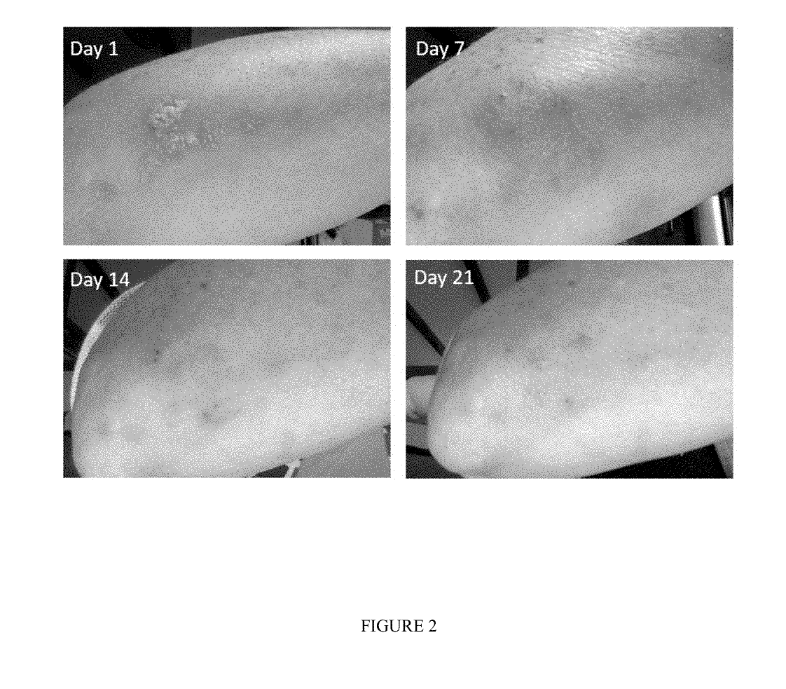

As with plaque #1, this psoriatic plaque became visibly smoother and flatter within the first week of treatment. Moreover, this plaque showed further improvement by Day 14 and was mostly resolved by Day 21, when treatment was stopped. The treatment was well tolerated and only some mild local irritation was observed. Photographs taken in weekly intervals are shown in FIG. 2.

Moreover, these plaques were also observed to remain in complete remission even 20 weeks after last treatment application. Indeed, as of the date of the last visit, approximately 8 months after the last treatment application with the exemplary formulation, this plaque remains in complete remission.

Plaque #3, Right Forearm

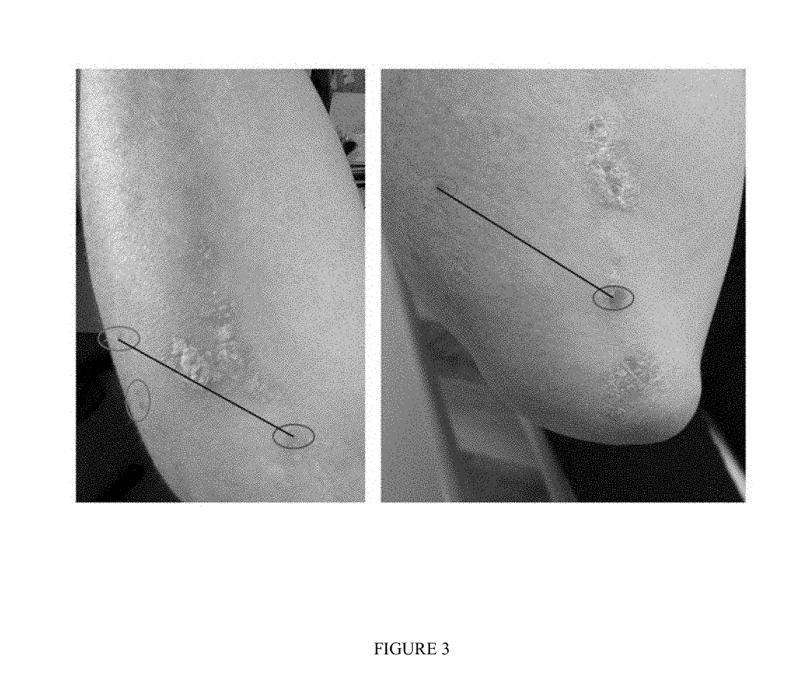

Approximately 5 months after Plaque #2 resolved, a new plaque evolved, lateral to the initial treatment site where the exemplary formulation of 3-BP was administered. As illustrated in FIG. 3, the original plaque #2 is shown on the left panel and the newly evolved Plaque #3 is seen on the right. Using landmarks of the skin clearly shows the difference in location.

Plaque #3 was treated with one drop per day at a concentration of 1.75 mM for one week. While the plaque improved, itching and pin-head sized crusting was also observed. Treatment was paused for one week and then resumed at 6 mM applied every other day. However, local irritation became more pronounced and treatment was stopped. Irritation resolved without sequelae, however, psoriatic skin afflictions remained.

Seven weeks later, another treatment attempt was started using 3-BP formulated at 1 mM, QD, for 10 days, which caused minor local irritation. Subsequently, dosing was reduced to 0.5 mM, applied once per day, which was well tolerated. Significant improvement in the severity of the psoriasis plaques was observed.

Plaque #4, Left Forearm

Approximately 24 weeks after the last treatment application of an exemplary formulation of 3-BP to Plaque #1 on the left forearm, a small, approximately 5 mm diameter, plaque appeared at the margin of the original treatment area (Plaque #4). This new area was treated with an exemplary formulation of 3-BP at 0.15 mM, QD, for 2.5 weeks. The treatment was well tolerated, however, showed only marginal efficacy. Between Day 18 and 43, a strength of 0.5 mM of an exemplary formulation of 3-BP was used. This concentration did not induce any local irritancy or other unwanted effects. Continued treatment completely resolved this plaque (Plaque #4), and was observed to remain resolved even more than 6 weeks after the last application.

Plaque #5, Left Elbow

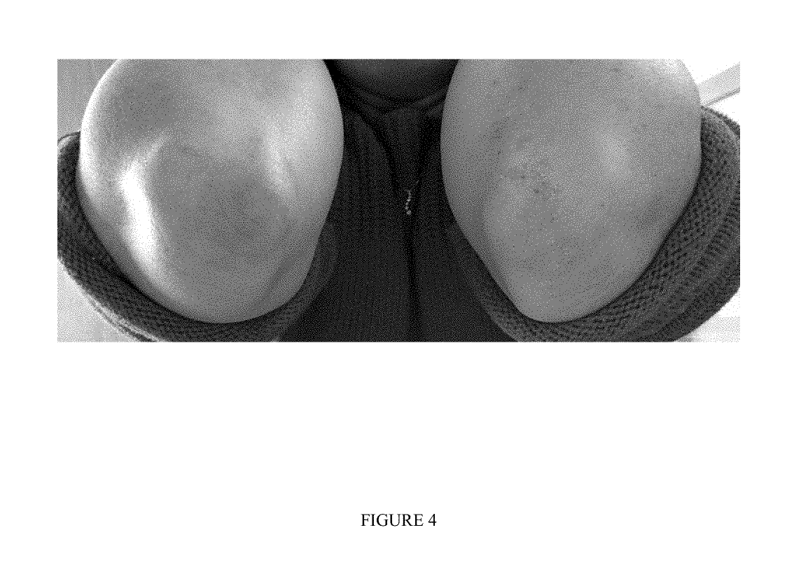

Predilection sites for psoriasis are on the extensor sides of limbs and, therefore, plaques located on elbows and knees are notoriously difficult to treat. Plaque #5, located on the left elbow of the subject, was treated for 10 days with an exemplary formulation of 3-BP at 1 drop per day at a strength of 0.15 mM. The treatment was well tolerated, but only marginal improvement was initially observed. From Days 10-19, an exemplary formulation of 3-BP at 0.5 mM was applied as 1 drop QD. Given the size of the treatment area, volume was increased to 2 drops QD starting Day 20. An exemplary image of treatment efficacy is shown in FIG. 4. The image in FIG. 4, taken on Day 47, shows the left (treated) elbow in comparison to the right (untreated). Notably, the left elbow is completely clear of psoriasis while there are minor psoriatic afflictions visible on the right elbow.

Summary of an Exemplary Case Report

In summary, an exemplary formulation of 3-BP at was found to be an effective and well tolerated. As described in the case study above, five different or recurring psoriasis plaques were been treated with a topical formulation of 3-BP. All treated plaques responded favorably or completely resolved over a period of three to six weeks. With the existing formulation as disclosed, the minimum effective dose appears to be near 0.15 mM, applied once per day. A strength of 0.5 mM was effective without any observable adverse effects. Concentrations of 1.0 mM and greater induced local irritation, including oozing and crusting. These adverse effects were generally painless but might be associated with itching at times. All unwanted effects resolved without sequelae, such as scarring or discoloration, within a few weeks after cessation of treatment.

For the exemplary formulation as used in this case study, a minimum effective dose was around 0.15 mM was observed; formulations of 0.5 mM were highly effective; and adverse events started to emerge at strengths of 1.0 mM or higher. Other formulations may have better or worse bio-delivery of the active ingredient and, therefore, the therapeutic window may be shifted.

Also, given that the first plaque being treated remains in full remission after more than eight months, a certain level of local side effects may be acceptable in order to fully eradicate the psoriatic skin cells and induce long lasting remissions.

The exemplary 3-BP formulation described above was formulated with a pH of approximately pH 3 and stored at low temperature to preserve stability of the compound. This exemplary formulation was of low viscosity and filled in ophthalmic dropper bottles. This packaging configuration provides for excellent dose volume control with each drop consistently being 25-50 μl. Dose-volume consistency may be advantageous for products with steep dose-response relationships and a narrow therapeutic window. However, greater volume control and consistency of low viscosity liquids have to be weighed against the risks of run-off and unintended exposure to off target sites.

It is envisioned that formulations that provide stability at room temperature and can be stored extended periods of time may be beneficial. Without wishing to be bound by theory, such formulations of an agent that targets energy production (e.g., 3-BP) may include low pH, anhydrous bases such as gels or ointments, slow-release formulations, encapsulation (such as cyclo-dextrane), or a combination of other such stability preserving approaches.

### Example 3

The present Example describes use of a topical formulation of 3-BP in the treatment of warts or other dysplastic conditions such as cervical dysplasia. The goal of this study is to completely eliminate/kill the dysplastic cells. Therefore, treatment is planned in a sequential dose escalating fashion until the maximum tolerated dose is established. Once local irritation is observed, dosing is continued at approximately the same level for up to four weeks.

The research is conducted as an out-patient, open label, uncontrolled experiment involving up to 10 subjects. Within each treatment area, the efficacy is estimated by visual inspection or microscopic swabbing, as appropriate. In addition, warts may be photographed and adjudicated by a blinded independent investigator. These efficacy variables are assessed at every visit.

Safety is assessed by collection of research events and review of concomitant treatments. Clinical adverse event reporting is conducted at each visit. Vital signs including weight, height, temperature, resting pulse, blood pressure, and respiration rate are recorded.

### Example 4

The present Example describes use of a topical formulation of 3-BP in the treatment of atopic dermatitis or eczema. The goal of this study is to reduce activity of pro-inflammatory cells of the immune system including mast cells, lymphocytes, granulocytes and others. Therefore, treatment is planned in a sequential dose escalating fashion avoiding additional local irritation. Dose escalation is accomplished by a step wise increase in formulation strength and/or application volume and frequency.

The research is conducted as an out-patient, open label, intra-individually controlled repeated measurement experiment involving up to 10 subjects. Eczema typically affects the flexor side of elbows or knees; to minimize bias, subjects are treated at one side only with the other side serving as an intra-individual control.

Within each treatment area, the severity is estimated by a severity score which is the sum of the intensity scores for four signs. The four signs are: redness (erythema, inflammation), thickness (induration, papulation, swelling—acute eczema), scratching (excoriation), and lichenification (lined skin, prurigo nodules—chronic eczema).

Safety is assessed by collection of research events and review of concomitant treatments. Clinical adverse event reporting is conducted at each visit. Vital signs including weight, height, temperature, resting pulse, blood pressure, and respiration rate are recorded.

Having thus described several aspects of at least one embodiment of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

### EQUIVALENTS

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

1. A method for treating or preventing reoccurrence of a skin disorder, the method comprising a step of:
administering a therapeutically effective amount of an agent that inhibits cellular energy production to the skin of a subject in need of such treatment, wherein the skin disorder is associated with increased cell activation including cell proliferation.

2. The method of claim 1, wherein the agent is characterized by an ability to reduce ATP production in cells found in the skin of the subject.

3. The method of claim 1, wherein the agent is selected from the group consisting of 2-deoxyglucose, 3-bromopyruvate, oxamate, apoptolidin, and combinations thereof.

4. The method of claim 1, wherein the agent is selected from the group of compounds represented by the general formula: 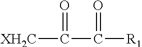
wherein X represents a halide, a sulfonate, a carboxylate, an alkoxide, or an amine oxide; X is a halide selected from the group consisting of: fluoride, bromide, chloride, and iodide.

5. The method of claim 1, wherein the agent is a glycolysis inhibitor.

6. The method of claim 5, wherein the glycolysis inhibitor is a glyceraldehyde-3-phosphate dehydrogenase inhibitor.

7. The method of claim 5, wherein the glycolysis inhibitor is a hexokinase inhibitor.

8. The method of any one of claims 4-7, wherein the agent is 3-bromopyruvate.

9. The method of any one of claims 1-8, wherein the activated skin disorder is selected from the group consisting of psoriasis, eczema, warts, including genital warts, hyperkeratosis, ichthyosis, keratoderma, lichen planus, and cervical dysplasia.

10. The method of claim 9, wherein the activated skin disorder is psoriasis.

11. The method of claim 9, wherein the activated skin disorder is eczema or atopic dermatitis.

12. The method of claim 1, wherein the agent that inhibits cellular energy production is administered in combination with another agent selected from: antibiotics, retinoids, anti-inflammatory agents, immunosuppressive agents, corticosteroids, vitamin A, vitamin A derivatives, vitamin D, and vitamin D analogs.

13. The method of claim 1, wherein the agent that inhibits cellular energy production is formulated for topical administration.

14. The method of claim 13, wherein the agent that inhibits cellular energy production is formulated as a spray, mist, aerosol, solution, lotion, gel, cream, ointment, paste, unguent, emulsion or suspension.

15. A method for treating or preventing reoccurrence of the activated skin disorder such as psoriasis, comprising administering a therapeutically effective amount of an anti-glycolytic agent to the skin of a subject in need of such treatment.

16. The method of claim 15, wherein the anti-glycolytic agent is a 3-halopyruvate selected from the group consisting of: 3-fluoropyruvate, 3-chloropyruvate, 3-bromopyruvate and 3-iodopyruvate.

17. The method of claim 15, wherein the anti-glycolytic agent is formulated for topical administration.

18. The method of claim 15, wherein the agent is characterized by an ability to reduce ATP production in skin cells.

19. A topical composition for treating or preventing the symptoms of psoriasis comprising an anti-glycolytic agent and a pharmaceutically acceptable carrier.

20. The topical composition of claim 19, wherein the anti-glycolytic agent is selected from the group of compounds represented by the general formula: 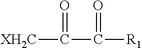
wherein X represents a halide, a sulfonate, a carboxylate, an alkoxide, or an amine oxide; X is a halide selected from the group consisting of: fluoride, bromide, chloride, and iodide.

21. The topical composition of claim 20, wherein the anti-glycolytic agent is 3-bromopyruvate or a pharmaceutically acceptable salt thereof.

22. The topical composition of any one of claims 19-21, wherein the anti-glycolytic agent is present in an amount in the range of about 0.0001 percent to about 50 percent of the total weight of the formulation.

23. The topical composition of any one of claims 19-22, wherein the pH value of the composition is in the range of about 2.5 to 8.

24. The topical composition of any one of claims 19-23, wherein the pharmaceutically acceptable carrier is an aqueous gel comprising water and a pharmaceutically acceptable gelling agent selected from the group consisting of: carbomers, glycerine polyacrylate and mixtures thereof.

25. The topical composition of any one of claims 19-23, wherein the pharmaceutically acceptable carrier is a cream or an ointment comprising: stearic acid, stearyl alcohol, cetyl alcohol, glycerin, and water.

26. The topical composition of any one of claims 19-25, further comprising a preservative.

27. The topical composition of any one of claims 19-26, further comprising a local anesthetic.

28. The topical composition of any one of claims 19-27, further comprising a skin humectant.

29. The topical composition of any one of claims 19-28, anti-glycolytic agent is provided in a concentration sufficient to decrease glucose metabolism in the skin of a subject treated with the topical composition.

30. A method of manufacturing a medicament for topical delivery of an agent for the treatment or prevention of psoriasis, comprising combining an agent that inhibits ATP production and a pharmaceutically acceptable carrier.

31. The method of manufacturing a medicament of claim 30, wherein the agent that inhibits ATP production is an anti-glycolytic agent.

32. The method of manufacturing a medicament of claim 30, wherein the agent that inhibits ATP production is 3-bromopyruvate.
